(19) United States
(12) Patent Application Publication
(10) Pub. No.: US 2024/0085403 A1
(43) Pub. Date: Mar. 14, 2024

(54) METHOD FOR INHIBITING ADVENTITIOUS VIRAL INFECTION

(71) Applicant: Allogene Therapeutics, Inc., South San Francisco, CA (US)

(72) Inventors: Thomas Charles PERTEL, San Mateo, CA (US); Ren SONG, San Mateo, CA (US); Diego A. VARGAS-INCHAUSTEGUI, Montara, CA (US); Garima YAGNIK, Walnut Creek, CA (US); Houman DEHGHANI, San Francisco, CA (US); Wenjing LI, Redwood City, CA (US); Jose PENA, Alameda, CA (US)

(21) Appl. No.: 18/450,554

(22) Filed: Aug. 16, 2023

Related U.S. Application Data
(60) Provisional application No. 63/510,025, filed on Jun. 23, 2023.
(60) Provisional application No. 63/398,364, filed on Aug. 16, 2022.

(51) Int. Cl. G01N 33/50; C12N 5/0783; C12N 7/00; C12Q 1/6851; C12Q 1/70

(52) U.S. Cl. CPC G01N 33/5023; C12N 5/0636; C12N 7/00; C12Q 1/6851; C12Q 1/705; C12N 2501/24; C12N 2501/51; C12N 2501/515; C12N 2710/16521

(57) ABSTRACT

The instant disclosure relates to methods for preventing, controlling and/or inhibiting the rare events of adventitious viral infection or viral replication/amplification after reactivation of latent viral infection during cell culture in the manufacturing process of cell-based drug products, including CAR T cell drug products. Also provided are in vitro cell culture models for HHV-6 latent infection and methods of making the same.

### CROSS REFERENCE TO RELATED APPLICATIONS

The present application claims the benefit of priority to U.S. Provisional Application No. 63/398,364, filed on Aug. 16, 2022, and U.S. Provisional Application No. 63/510,025, filed on Jun. 23, 2023, the contents of both of which are hereby incorporated by reference in their entireties.

### REFERENCE TO SEQUENCE LISTING

The instant application contains a Sequence Listing which has been submitted electronically in XML file format and is hereby incorporated by reference in its entirety. Said XML copy, created on Aug. 14, 2023, is named AT-055-03US_ST26.xml and is 37,606 bytes in size.

### FIELD

The instant disclosure relates to methods for preventing and/or inhibiting adventitious viral infection and preventing and/or inhibiting the rare events of reactivation of infection after reactivation of latent viral infection, during extended cell culture such as extended culture of primary cells with latent viral infection in the manufacturing of cell-based drug products, including chimeric antigen receptor (CAR) T cell drug products.

### BACKGROUND

Human herpesvirus 6 (HHV-6) commonly infects humans at a young age. HHV-6 can be divided into two closely related but distinct subtypes, HHV-6A and HHV-6B. Both subtypes can establish lytic infection and latent infection and both subtypes can infect human T cells. HHV-6B accounts for the great majority of infection in the United States, UK and Japan, whereas HHV-6A infections are largely confined to sub-Saharan Africa. HHV-6B is the etiologic agent of roseola infantum and is associated with neurological diseases, such as encephalitis. The epidemiology and symptoms associated with HEINZ-6A remain poorly defined. The specific triggers of reactivation of both strains of HHV-6 are not well-understood. The lack of a cell culture model for HHV-6 latent infection and reactivation has hampered such research in the field. For living cell-based therapies, such as stem cell transplant therapies, and autologous and allogeneic CAR T cell-based therapies, adventitious viral infection, though rare, may occur during cell culture in the manufacturing processes. Furthermore, reactivation of viruses that can establish latent infection, such as HHV-6, may also occur, though extremely rare. In allogeneic cell-based therapies, healthy donors are screened for transmissible and communicable diseases to minimize the risk in the downstream manufacturing process. In addition, adventitious viral infection and reactivation of endogenous or endemic viruses during cell culture in the manufacturing process can be detected by stringent screening assay as part of the quality control process to ensure drug safety. Any drug product batches that fail the safety standard will be rejected.

Therefore, there exists a need for methods for preventing and controlling the rare events of adventitious viral infection or infection after reactivation during the manufacturing process of cell-based therapies to ensure drug safety and minimize waste. There also exists a need for an established cell culture model for HHV-6 latent infection and reactivation.

### SUMMARY

Provided herein are methods of making engineered immune cells, or methods for inhibiting and/or controlling the rare events of adventitious viral infection and/or viral reactivation during cell culture in the manufacturing process of cell-based therapies, including CAR T cell therapies.

In one aspect, provided herein are methods of preventing, controlling or inhibiting HHV-6 replication after reactivation of isolated immune cells in cell culture comprising the step of contacting the immune cells with an antiviral agent in a culture medium. In certain embodiments, the antiviral agent is interferon (IFN), e.g., IFNα, ganciclovir, cidofovir or foscarnet, or a salt thereof. In another aspect, provided herein are methods of preventing, controlling or inhibiting HHV-6 replication, preventing, controlling or inhibiting HHV-6 transcription activation, preventing, controlling or inhibiting HHV-6 infection, or preventing, controlling or inhibiting HHV-6 replication or infection after reactivation, in a process of making engineered immune cells, the method comprising the steps of culturing immune cells, engineering the immune cells, and contacting the immune cells with an anti-viral agent in a culture medium. In certain embodiments, the antiviral agent is IFNα, foscarnet, ganciclovir, or cidofovir.

In some embodiments, the HHV-6 is human HHV-6A. In certain embodiments, the HHV-6 is human HHV-6B.

In some embodiments, the step of contacting the immune cells with the antiviral agent occurs by adding the antiviral agent to the culture medium. In certain embodiments, the antiviral agent is added to the culture medium on day 4 to day 10, day 5 to day 10, day 6 to day 10, day 7 to day 10, day 8 to day 10, or day 9 to day 10 of the process of making the engineered immune cells. In some embodiments, the antiviral agent is added to the cell culture medium after the cells have been cultured for at least 4 days, at least 5 days, at least 6 days, at least 7 days, at least 8 days, or at least 9 days. In some embodiments, the immune cells are contacted with the antiviral agent for about 1 day, about 2 days, about 3 days, about 4 days, or about 5 days. In some embodiments, the immune cells are PBMCs. In some embodiments, the immune cells are T cells. In some embodiments, the immune cells are activated on day 1 of the process of making the engineered immune cells, optionally after the immune cells have been thawed. In some embodiments, the immune cells are activated by contacting the immune cells with anti-CD3 and anti-CD28 antibodies. In some embodiments, the antiviral agent is added to the cell culture medium 3 days, 4 days, 5 days, 6 days, 7 days, or 8 days after the immune cells have been activated. In some embodiments, the antiviral agent is IFN. In some embodiments, the IFN is a type I IFN or a type III IFN. In some embodiments, the IFN is IFNα. In some embodiments, the IFN, e.g., IFNα, is added to the culture medium at a concentration of about 0.1 ng/ml to about 100 ng/ml, about 0.1 ng/ml to about 10 ng/ml, or about 0.1 ng/ml to about 1 ng/ml. In some embodiments, the antiviral agent is foscarnet, or a salt thereof. In some embodiments, the foscarnet, or a salt thereof, is added to the culture medium at a concentration of about 8 μM to about 20 μM, about 8 μM to about 15 μM, about 8 μM, about 9 μM, about 10 μM, about 11 μM, about 12 μM, about 13 μM, about 14 μM, or about 15 μM. In some embodiments, the antiviral is ganciclovir, or a salt thereof. In some embodiments, the ganciclovir, or a salt thereof, is added to the culture medium at a concentration of about 25 μM to about 35 μM, about 25 μM to about 30 μM, about 27 μM to about 30 μM, about 25 μM, about 26 μM, about 27 μM, about 28 μM, about 29 μM, about 30 μM, or about 35 μM. In some embodiments, the antiviral is cidofovir, or a salt thereof. In some embodiments, the cidofovir, or a salt thereof, is added to the culture medium at a concentration of about 14 μM to about 25 μM, about 14 μM to about 20 μM, about 14 μM, about 15 μM, about 16 μM, about 17 μM, about 18 μM, about 19 μM, or about 20 μM.

In some embodiments, the immune cells are T cells, PBMCs, IPSCs or NK cells. In some embodiments, the immune cells are obtained from a patient or from a healthy donor. In some embodiments, the immune cells harbor latently infected HHV-6 genome or are latently infected by HHV-6.

In some embodiments, the immune cells exhibit reduced levels of HHV-6 replication, HHV-6 transcription activation, HHV-6 infection, or HHV-6 replication or infection after reactivation, as compared to control immune cells without being contacted with the antiviral agent. In some embodiments, the immune cells exhibited comparable levels of combined Tcm and Tscm as compared to control immune cells without being contacted with the antiviral agent. In some embodiments, the method further comprises the step of detecting or measuring HHV-6 DNA levels, RNA levels or protein levels during the process of making engineered immune cells. In some embodiments, the HHV-6 DNA levels, RNA levels, and/or protein levels are measured on day 1, day 2, day 3, day 4, day 5, day 6, day 7, day 8, day 9, day 10, day 11, day 12, day 13, day 14, day 15, day 16, day 17, day 18, day 19, day 20, or after day 20 of the process of making the engineered immune cells. In some embodiments, the HHV-6 DNA levels, RNA levels, and/or protein levels are measured at the beginning of, and/or at the end of, the process of making the engineered immune cells. In some embodiments, the HHV-6 DNA levels, RNA levels, and/or protein levels measured at the beginning of the process of making the engineered immune cells and at the end of the process of making the engineered immune cells are comparable. In some embodiments, the HHV-6 DNA levels, RNA levels, and/or protein levels measured on about day one to about day 5 of the process of making the engineered immune cells are comparable to the levels measured on about day 15 to about day 20 of the process of making the engineered immune cells. In some embodiments, the method further comprises the step of detecting HHV-6 RNA levels during the process of making the engineered immune cells, wherein HHV-6 RNA levels are not detectable. In some embodiments, the HHV-6 DNA levels, RNA levels or protein levels are determined by PCR, qPCR, RT-PCR, RT-qPCR, ELISA, immunofluorescent assay, or flow cytometry.

In some embodiments, the antiviral agent is IFN, and preferably the IFN is IFNα. In certain embodiments, the IFN is human IFNα.

Also provide herein are methods of making engineered immune cells comprising the steps of (a) culturing immune cells; (b) engineering the immune cells; and (c) contacting the immune cells with an antiviral agent, e.g., interferon (IFN), in a culture medium, wherein the immune cells comprise latent HHV-6 infection or are latently infected with HHV-6. In some embodiments, the step of contacting the immune cells with the antiviral agent occurs by adding the antiviral agent to the culture medium. In some embodiments, the antiviral agent is added to the culture medium on day 4 to day 10, day 5 to day 10, day 6 to day 10, day 7 to day 10, day 8 to day 10, day 9 to day 10 after culturing the immune cells. In some embodiments, the antiviral agent is added to the cell culture medium after the cells have been cultured for at least 4 days, at least 5 days, at least 6 days, at least 7 days, at least 8 days, or at least 9 days. In some embodiments, the immune cells are activated on day 1 of the process of making the engineered immune cells, optionally after the immune cells have been thawed. In some embodiments, the immune cells are activated by contacting the immune cells with anti-CD3 and anti-CD28 antibodies. In some embodiments, the antiviral agent is added to the cell culture medium 3 days, 4 days, 5 days, 6 days, 7 days, or 8 days after the immune cells have been activated. In some embodiments, the immune cells are contacted with the antiviral agent for about 1 day, about 2 days, about 3 days, about 4 days, or about 5 days. In some embodiments, the antiviral agent is IFN, e.g., a type I IFN or a type III IFN, foscarnet, ganciclovir, or cidofovir, or a salt thereof. In some embodiments, the IFN is human IFNα. In some embodiments, the antiviral agent is or comprises IFN that is added to the culture medium at a concentration of about 0.1 ng/ml to about 100 ng/ml, about 0.1 ng/ml to about 10 ng/ml, or about 0.1 ng/ml to about 1 ng/ml. In some embodiments, the IFN is IFNα. In certain embodiments, the IFN is human IFNα. In some embodiments, the antiviral agent is or comprises foscarnet, or a salt thereof. In some embodiments, the foscarnet, or a salt thereof, is added to the culture medium at a concentration of about 8 μM to about 20 μM, about 8 μM to about 15 μM, about 8μM, about 9 μM, about 10 μM, about 11 μM, about 12 μM, about 13 μM, about 14 μM, or about 15 μM. In some embodiments, the antiviral is or comprises ganciclovir, or a salt thereof. In some embodiments, the ganciclovir, or a salt thereof, is added to the culture medium at a concentration of about 25 μM to about 35 μM, about 25 μM to about 30 μM, about 27 μM to about 30 μM, about 25 μM, about 26 μM, about 27 μM, about 28 μM, about 29 μM, about 30 μM, or about 35 μM. In some embodiments, the immune cells are T cells, PBMCs, iPSCs or NK cells. In some embodiments, the immune cells are obtained from a patient or a healthy donor.

In some embodiments, the method further comprises the step of detecting or measuring HHV-6 DNA levels, RNA levels or protein levels after the step of contacting the immune cells with the antiviral agent. In some embodiments, the method further comprises a step of detecting or measuring HHV-6 DNA levels before contacting the immune cells with the antiviral agent, wherein the HHV-6 DNA levels measured after contacting the immune cells with the antiviral agent are comparable to the HHV-6 DNA levels measured before the step of contacting the immune cells with the antiviral agent.

In some embodiments, the step of engineering the immune cells comprises introducing to the immune cells an exogenous polynucleotide that encodes a chimeric antigen receptor (CAR) or recombinant T cell receptor (TCR). In some embodiment, the engineered immune cells are CAR T cells. In some embodiments, the exogenous polynucleotide is introduced to the immune cells by lentiviral transduction or by adenovirus associated viral transduction. In some embodiments, the step of engineering the immune cells further comprises modifying one or both TCRα genetic loci to reduce or eliminate the expression or activity of the TCRα gene.

In another aspect, provided herein are an engineered immune cell, or a population of engineered immune cells, produced by the method described herein.

In a further aspect, the instant disclosure provides methods for generating an in vitro cell culture model of HHV-6 latent infection and reactivation comprising the steps of (a) infecting human lymphoid cells with HHV-6; (b) culturing the infected cells for about 12 to about 19 days or until logarithmic growth of the virus is observed; and (c) serially passaging the cells to maintain a cell density of about 0.2-0.8×10⁶ cells per cm² for about 30 days to about 60 days, thereby establishing latent HHV-6 infection in the cells. In some embodiments, the method further comprises the step of detecting HHV-6 viral DNA or detecting HHV-6 viral RNA and/or HHV-6 protein, after serial passaging of the cells in step (c), wherein a constant level of HHV-6 DNA or an absence of detectable RNA and/or protein indicates latent infection. In some embodiments, the latent HHV-6 infection in the cells can be reactivated by a stimulant. In some embodiments, the stimulant is sodium butyrate and/or PMA (phorbol-12-myristate-13-acetate). In some embodiments, the method further comprises a step of detecting expression of one or more viral transcripts, wherein detection of the one or more viral transcripts is indicative of viral reactivation.

In a related aspect, the instant disclosure also provides an in vitro cell culture model for HHV-6 latent infection and reactivation generated by the method described herein, wherein no more than 10⁴ copies of HHV-6 viral genome equivalents per 500 ng extracted genomic DNA can be detected by qPCR in the cells and/or no HHV-6 transcript can be detected by RT-qPCR in the cells. In some embodiments, active HHV-6 infection can be induced or reactivated in the cells, optionally by a stimulant. In some embodiments, the stimulant comprises sodium butyrate and PMA. In some embodiments, active HHV-6 infection can be demonstrated by detection of one or more HHV-6 transcripts associated with viral functional or structural genes, optionally by RT-PCR or RT-qPCR.

### BRIEF DESCRIPTION OF THE DRAWINGS

FIG. 1A shows a bar graph of the levels of HHV-6 viral DNA in selected CAR T drug products, and FIG. 1B lower panels show images of immunofluorescence staining of the levels of HHV-6 p41 protein, with or without IFNα treatment. The top panels of FIG. 1B show image of DAPI staining for nuclei. The data in FIG. 1C show longitudinal analysis of HHV-6 U31 DNA levels of two batches of CAR T cell culture.

FIG. 2A depicts the experimental design for establishing a cell culture model of HHV-6 latent infection. FIG. 2B shows U31 or U65-66 DNA levels in cells during the initial 19 days post infection (left panel), and the DNA levels in cells in the continuous culture after serial passaging two months later (Time point 1) and then one week later (Time point 2) (right panel). The DNA levels were measured by qPCR. FIG. 2C shows the absence of viral transcripts during the continuous cell culture as determined by using the nCounter® system. FIGS. 2D-2E show the increased viral DNA levels (FIG. 2D) and increased viral transcripts (FIG. 2E) detected upon reactivation. IE: viral immediate early genes; IE-E: viral immediate early-early genes; E: viral early genes; and L: viral late genes. FIG. 2F shows the increased viral transcripts detected over-time post reactivation-inducing treatment (Tx).

FIGS. 3A-3B show that reactivation resulted in increased viral DNA levels in the cell culture latency model (FIG. 3A) and that increased IFNα added either before or after reactivation suppressed viral DNA amplification (FIG. 3B).

FIG. 4A depicts the experiment design testing the effects of IFNα on HHV-6 infection in a small-scale allogeneic CAR T production process using cells from two donors. FIG. 4B shows the fold exchange of viral gene U79, U90, and U100 RNAs as determined by RT-qPCR in the presence or absence of IFNα with or without HHV-6 infection from Day 11-Day 18 (i.e., 3 days to 10 days post infection). Viral protein p41 and viral gene U31 DNA levels were measured under the same conditions and the results are shown in FIG. 4C and FIG. 4D, respectively.

The data in FIG. 5A examined the cell expansion fold and percentage CAR+ from Day 8 to Day 18 of the small-scale CAR T production process (i.e., 0-10 days post HHV-6 infection). FIG. 5B examined the cell phenotype of CAR+ cells on Day 18 (i.e., 10 days post HHV-6 infection) under different treatment conditions.

The results in FIGS. 6A-6B show relative-fold changes of HHV-6 U100 transcript (FIG. 6A) or U79 transcript (FIG. 6B) as compared to control in samples taken on different days in the process under different IFNα treatment conditions. FIGS. 7A-7B are the same experiments repeated in cells from a different donor.

FIG. 8 depicts results of apoptosis analysis of iCiHHV6 cells treated with different antiviral agents on day 3 or day 15 post antiviral treatment. The data in FIG. 9 show the HHV-6 levels as determined by U65 gene copies analyzed by qPCR.

### DETAILED DESCRIPTION

In one aspect, the instant disclosure provides methods of preventing, controlling or inhibiting HHV-6 infection or replication after HHV-6 reactivation of isolated immune cells in cell culture comprising the step of contacting the immune cells with an antiviral agent, e.g., IFN, in a culture medium.

In one particular aspect, provided herein are methods of preventing, controlling or inhibiting HHV-6 amplification, replication, infection, and/or transcriptional activation comprising contacting immune cells with an antiviral agent, e.g., IFN. One application is to the manufacturing process of engineered immune cells, such as CAR T cells. By preventing the rare events of HHV-6 amplification or replication, for example after viral reactivation during extended cell culture in the manufacturing process, the rejection rate of clinical-grade lots can be further reduced.

As used herein, the terms “a” and “an” are used to mean one or more. For example, a reference to “a cell,” “an immune cell” or “an antibody” means “one or more cells,” “one or more immune cells” or “one or more antibodies.”

Type I and type III interferons (IFNs) are a large group of proteins that collectively regulate the activity of the human immune system. The IFN response constitutes the major first line of defense against viruses. Recognition of viral infections by innate immune sensors activates type I and type III IFN responses. Human type I IFNs includes IFNα, IFNβ, IFNε, IFNκ, IFNω, and type III IFN includes IFNλ. IFNs are antiviral agents, which activate immune cells, including leukocytes and T cells, and NK cells, and modulate functions of the immune system. IFN is released by viral infected cells and exerts its function through binding to the cell surface receptors and activating many interferon-stimulated genes (ISGs), which have the capacity to interfere with every step of viral replication. See Park & Iwasaki, 2020, Cell Host & Microbe, 27:870-878.

It has not, however, been demonstrated that IFN can be used as a culture media additive that can block in process HHV-6 infection, replication, infection or viral replication/amplification after viral reactivation, and/or transcriptional activation during the culturing or manufacturing of immune cells, or during the culturing or manufacturing of engineered immune cells. In certain embodiments, the immune cells or engineered immune cells are autologous CAR T cells. In certain embodiments, the immune cells or engineered immune cells are allogeneic CAR T cells.

In some embodiments, the IFN is a type I IFN. In certain embodiments, the IFN is IFNα or IFNβ. In some embodiments, the IFN is added to the culture medium at a concentration of about 0.01 ng/ml to about 100 ng/ml, about 0.01 ng/ml to about 10 ng/ml, about 0.01 ng/ml to about 1 ng/ml, about 0.01 ng/ml to about 0.1 ng/ml, about 0.1 ng/ml to about 100 ng/ml, about 0.1 ng/ml to about 10 ng/ml, about 0.1 ng/ml to about 1 ng/ml, about 0.1 ng/ml to about 0.9 ng/ml, about 0.1 ng/ml to about 0.8 ng/ml, about 0.1 ng/ml to about 0.7 ng/ml, about 0.1 ng/ml to about 0.6 ng/ml, about 0.1 ng/ml to about 0.5 ng/ml, about 0.1 ng/ml, about 0.2 ng/ml, about 0.3 ng/ml, about 0.4 ng/ml, about 0.5 ng/ml, about 0.6 ng/ml, about 0.7 ng/ml. about 0.8 ng/ml, about 0.9 ng/ml, or about 1 ng/ml. In certain embodiments, the IFN is IFNα. In certain embodiments, the IFN is human IFNα.

Although currently no treatment has been officially developed for HHV-6, several existing antiviral agents, including foscarnet, ganciclovir, and cidofovir, have been shown in vitro efficacy against HHV-6. See hhv-6foundation.org/research/hhv-6-antiviral-drug-resistance. However, it has not been shown that they can be used as a culture media additive that can block in process HHV-6 infection, replication, infection or viral replication/amplification after viral reactivation, and/or transcriptional activation during the culturing or manufacturing of immune cells, or during the culturing or manufacturing of engineered immune cells. In some embodiments, the antiviral agent is foscarnet, or a salt thereof. In some embodiments, the foscarnet, or a salt thereof, is added to the culture medium at a concentration of about 8 μM to about 20 μM, about 8 μM to about 15 μM, about 8 μM, about 9 μM, about 10 μM, about 11 μM, about 12 μM, about 13 μM, about 14 μM, or about 15 μM. In some embodiments, the antiviral is ganciclovir, or a salt thereof. In some embodiments, the ganciclovir, or a salt thereof, is added to the culture medium at a concentration of about 25 μM to about 35 μM, about 25 μM to about 30 μM, about 27 μM to about 30 μM, about 25 μM, about 26 μM, about 27 μM, about 28 μM, about 29 μM, about 30 μM, or about 35 μM.

The instant disclosure provides methods of preventing or controlling HHV-6 replication of isolated immune cells or engineered immune cells. In some embodiments, the HHV-6 replication can be a result of viral infection, viral spreading after reactivation of latent viral infection (latency). In some embodiments, the methods disclosed herein prevent the progression of active infection. In some embodiments, the methods disclosed herein prevent progression (or spread) of virus infection following reactivation of latent viral infection (or viral latency). In some embodiments, the methods disclosed herein prevent the reactivated latent viral infection from progressing to active infection. Accordingly, the methods disclosed herein inhibit HHV-6 replication of isolated immune cells or engineered immune cells. In some embodiments, the methods disclosed herein inhibit HHV-6 active infection of isolated immune cells or engineered immune cells. In certain embodiments, the immune cells or engineered immune cells are autologous CAR T cells or allogeneic CAR T cells.

The term “viral replication,” or “viral amplification” as used herein can refer to active viral infection as opposed to latent viral infection. Such an active infection may or may not result in virion production and/or lytic infection. For endemic viruses that can establish latent infection in the cells, an increase in viral DNA levels as compared to the levels of latent infection, and/or the detection of, or an increase in, one or more viral RNA transcripts associated with functional or structural genes as compared to the levels of latent infection, can be indicative of active infection, and thus indicative of viral replication or viral amplification. Accordingly, the instant disclosure provides methods of preventing, controlling or inhibiting HHV-6 replication or amplification after viral reactivation. Viral DNA levels and viral RNA transcript levels can be detected by methods known in the art and/or described herein. In some embodiments, the virus is HHV-6.

In some embodiments, HHV-6 replication can be determined by detecting the presence or increase of viral DNA, as measured by methods including, without limitations, PCR, qPCR, bulk DNA sequencing, or single cell DNA sequencing. In some embodiments, HHV-6 replication can be determined by detecting the presence or increase of viral RNA transcripts, as measured by methods, including without limitations, RT-PCR, RT-qPCR, nCounter®, bulk RNA sequencing or single cell RNA sequencing. In some embodiments, HHV-6 replication can be determined by detecting the presence or increase of viral protein expression, as measured by methods, including without limitations, flow cytometry, immunofluorescence staining or other immunology-based methods. In some embodiments, HHV-6 replication can be determined by detecting and/or quantifying cytopathic effects (CPE) resulted from viral replication. In some embodiments, HHV-6 replication can be determined by co-culturing the immune cells or engineered immune cells with a reporter cell line susceptible to HHV-6 infection, or exposing the reporter cell line to the cell culture supernatant of the immune cells or engineered immune cells, and detecting and/or quantifying HHV-6 infection of the reporter cell line according to methods known in the art and/or described herein.

In another aspect, the instant disclosure provides methods of preventing or inhibiting HHV-6 transcription activation. In some embodiments, HHV-6 transcription activation can be the activation of one or more HHV-6 immediate early genes, early genes and/or late genes transcription. In some embodiments, HHV-6 transcription activation indicates viral reactivation. In some embodiments, HHV-6 transcription activation indicates productive or active viral infection. In some embodiments, HHV-6 transcription activation can be determined by detecting the presence or increase of viral RNA transcripts, as measure by methods, including without limitations, RT-PCR or RT-qPCR.

In a related aspect, the instant disclosure provides methods of preventing, controlling or inhibiting HHV-6 replication.

Further, provided herein are methods of preventing, controlling or inhibiting HHV-6 replication, preventing, controlling or inhibiting HHV-6 transcription activation, preventing, controlling or inhibiting HHV-6 infection, or preventing, controlling or inhibiting HHV-6 replication or infection after reactivation, in a process of making engineered immune cells, the method comprising the steps of culturing immune cells, engineering the immune cells, and contacting the immune cells with IFN in a culture medium. Adventitious viruses, such as HHV-6, infect humans at a young age and establish endemic latent infections in the general population. The disclosure provides advantageous methods of preventing, controlling and/or inhibiting viral replication after reactivation of such endemic latent infections in ex vivo or in vitro cell culture in a process of producing cell-based therapies, though the reactivation event is exceedingly rare. The cell-based therapies can be autologous cell-based therapies that are derived, engineer, or obtained from a patient's immune cells. The cell-based therapies can be allogeneic cell-based therapies that are derived, obtained, or engineered from a healthy volunteer's immune cells.

In some embodiments, the cells are engineered immune cells. In some embodiments, the engineered immune cells are CAR T cells. In some embodiments, the engineered immune cells are CAR NK cells. In some embodiments, the engineered immune cells are allogeneic or autologous immune cells. In some embodiments, the engineered immune cells are autologous or allogeneic CAR T cells.

### Immune Cells

Cells suitable for use with the methods and/or reagents described herein include immune cells.

Prior to the in vitro manipulation or genetic modification (e.g., as described herein), cells for use in methods described herein (e.g., immune cells) can be obtained from a subject. Cells can be obtained from a number of non-limiting sources, including peripheral blood mononuclear cells (PBMCs), bone marrow, lymph node tissue, cord blood, thymus tissue, stem cell- or iPSC-derived immune cells, tissue from a site of infection, ascites, pleural effusion, spleen tissue, and tumors. In some embodiments, any number of T cell lines available and known to those skilled in the art, can be used. In some embodiments, cells can be derived from a healthy donor, from a patient diagnosed with cancer or from a patient diagnosed with an infection. In some embodiments, cells can be part of a mixed population of cells which present different phenotypic characteristics.

In some embodiments, immune cells are autologous immune cells obtained from a subject who will ultimately receive the engineered immune cells. In some embodiments, immune cells are allogeneic immune cells obtained from a donor, who is a different individual from the subject who will receive the engineered immune cells.

In some embodiments, immune cells comprise T cells. T cells can be obtained from a number of sources, including peripheral blood mononuclear cells (PBMCs), bone marrow, lymph nodes tissue, cord blood, thymus tissue, stem cell- or iPSC-derived T cells, tissue from a site of infection, ascites, pleural effusion, spleen tissue, and tumors. In some embodiments, T cells can be obtained from a volume of blood collected from the subject using any number of techniques known to the skilled person, such as FICOLL™ separation.

Cells can be obtained from the circulating blood of an individual by apheresis. The apheresis product typically contains lymphocytes, including T cells, monocytes, granulocytes, B cells, other nucleated white blood cells, red blood cells, and platelets. In some embodiments, the cells collected by apheresis can be washed to remove the plasma fraction, and placed in an appropriate buffer or media for subsequent processing.

PBMCs can be used directly for genetic modification with the immune cells (such as CARs or TCRs) using methods as described herein. In certain embodiments, after isolating the PBMCs, T lymphocytes can be further isolated and both cytotoxic and helper T lymphocytes can be sorted into naive, memory, and effector T cell subpopulations either before or after genetic modification and/or expansion.

In certain embodiments, T cells are isolated from PBMCs by lysing the red blood cells and depleting the monocytes, for example, using centrifugation through a PERCOLL™ gradient. A specific subpopulation of T cells, such as CCR7+, CD95+, CD122, CD27+, CD69+, CD127+, CD28+, CD3+, CD4+, CD8+, CD25+, CD62L+, CD45RA+, and CD45RO+ T cells can be further isolated by positive or negative selection techniques known in the art. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. Flow cytometry and cell sorting can also be used to isolate cell populations of interest for use in the present disclosure.

In some embodiments, a population of T cells is enriched for CD4+ cells.

In some embodiments, a population of T cells is enriched for CD8+ cells.

In some embodiments, a population of T cells is enriched for CD4+ and CD8+ cells.

In some embodiments, CD8+ cells are further sorted into naive, central memory, and effector cells by identifying cell surface antigens that are associated with each of these types of cells. In some embodiments the expression of phenotypic markers for naïve T cells include CD45RA+, CD95−, IL2Rβ−, CCR7+, and CD62L+. In some embodiments the expression of phenotypic markers for stem cell memory T cells include CD45RA+, CD95+, IL2Rβ+, CCR7+, and CD62L+. In some embodiments the expression of phenotypic markers for central memory T cells include CD45RO+, CD95+, IL2Rβ+, CCR7+, and CD62L+. In some embodiments the expression of phenotypic markers for effector memory T cells include CD45RO+, CD95+, IL2Rβ+, CCR7−, and CD62L−. In some embodiments the expression of phenotypic markers for T effector cells include CD45RA+, CD95+, IL2Rβ+, CCR7−, and CD62L−. Thus, CD4+ and/or CD8+ T helper cells can be sorted into naive, stem cell memory, central memory, effector memory and T effector cells by identifying cell populations that have cell surface antigens.

It will be appreciated that PBMCs can further include other cytotoxic lymphocytes such as NK cells or NKT cells. An expression vector carrying the coding sequence of a chimeric receptor as disclosed herein can be introduced into a population of human donor T cells, NK cells or NKT cells. Standard procedures are used for cryopreservation of T cells expressing the CAR for storage and/or preparation for use in a human subject. In one embodiment, the in vitro transduction, culture and/or expansion of T cells are performed in the absence of non-human animal derived products such as fetal calf serum and fetal bovine serum. In various embodiments a crypreservative media can comprise, for example, CryoStor® CS2, CS5, or CS10 or other medium comprising DMSO, or a medium that does not comprise DMSO.

### Engineered Immune Cells

Provided herein are engineered immune cells expressing the CARs of the disclosure (e.g., CAR-T cells).

In some embodiments, an engineered immune cell comprises a population of CARs, each CAR comprising extracellular antigen-binding domains. In some embodiments, an engineered immune cell comprises a population of CARs, each CAR comprising different extracellular antigen-binding domains. In some embodiments, an immune cell comprises a population of CARs, each CAR comprising the same extracellular antigen-binding domains.

The engineered immune cells can be allogeneic or autologous.

As used herein “autologous” means that cells, a cell line, or population of cells used for treating patients are originating from said patient.

As used herein “allogeneic” means that cells, a cell line, or population of cells used for treating patients are not originating from said patient but from a donor. In some embodiments, the donor is a healthy donor.

In some embodiments, the engineered immune cell is a T cell (e.g., inflammatory T-lymphocyte, cytotoxic T-lymphocyte, regulatory T-lymphocyte, helper T-lymphocyte, or tumor infiltrating lymphocyte (TIL)), NK cell, NK-T-cell, TCR-expressing cell, dendritic cell, killer dendritic cell, a mast cell, or a B-cell. In some embodiments, the cell can be derived from the group consisting of CD4+ T-lymphocytes and CD8+ T-lymphocytes. In some exemplary embodiments, the engineered immune cell is a T cell. In some exemplary embodiments, the engineered immune cell is an alpha beta T cell. In some exemplary embodiments, the engineered immune cell is a gamma delta T cell. In some exemplary embodiments, the engineered immune cell is a macrophage. In some embodiments, the engineered immune cells are human cells.

In some embodiments, the engineered immune cell can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells (iPSC), totipotent stem cells or hematopoietic stem cells. Stem cells can be CD34+ or CD34−.

In some embodiments, the cell is obtained or prepared from peripheral blood. In some embodiments, the cell is obtained or prepared from peripheral blood mononuclear cells (PBMCs). In some embodiments, the cell is obtained or prepared from bone marrow. In some embodiments, the cell is obtained or prepared from umbilical cord blood. In some embodiments, the cell is a human cell. In some embodiments, the cell is transfected or transduced by the nucleic acid vector using a method, including without limitation, electroporation, sonoporation, biolistics (e.g., Gene Gun), transfection, lipid transfection, polymer transfection, nanoparticles, viral transduction or viral transfection (e.g., retrovirus, lentivirus, AAV) or polyplexes. In some embodiments the cell is a T cell that has been re-programmed from a non-T cell. In some embodiments the cell is a T cell that has been re-programmed from a T cell.

### Binding Agents

In embodiments, the disclosed methods comprise the use of an antibody or antigen binding agent (e.g., comprising an antigen binding domain or comprising an antibody or fragment thereof). As discussed below, in various embodiments engineered immune cells can also comprise a binding agent.

As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CHI, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain. Those skilled in the art are well familiar with antibody structure and sequence elements, recognize “variable” and “constant” regions in provided sequences, and understand that there may be some flexibility in definition of a “boundary” between such domains such that different presentations of the same antibody chain sequence may, for example, indicate such a boundary at a location that is shifted one or a few residues relative to a different presentation of the same antibody chain sequence.

Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation.

For purposes of the instant disclosure, in certain embodiments, any polypeptide or complex of polypeptides that includes sufficient immunoglobulin domain sequences as found in natural antibodies can be referred to and/or used as an “antibody,” whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art.

Moreover, the term “antibody” as used herein, can refer to any of the art-known or developed constructs or formats for utilizing antibody structural and functional features in alternative presentation. For example, in some embodiments, an antibody utilized in the methods of the instant disclosure is in a format selected from, but not limited to, intact IgA, IgG, IgE or IgM antibodies; bi- or multi- specific antibodies (e.g., Zybodies®, etc.); antibody fragments such as Fab fragments, Fab fragments, F(ab)2 fragments, Fd fragments, and isolated CDRs or sets thereof; single chain variable fragments (scFVs); polypeptide-Fc fusions; single domain antibodies (e.g., shark single domain antibodies such as IgNAR or fragments thereof); camelid antibodies (also referred to herein as nanobodies or VHHs); shark antibodies, masked antibodies (e.g., Probodies®); Small Modular ImmunoPharmaceuticals (SMIPs™); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies;, Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.).

As used herein, the term “antibody agent” generally refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to, monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc. as is known in the art. In many embodiments, the term “antibody agent” is used to refer to one or more of the art-known or developed constructs or formats for utilizing antibody structural and functional features in alternative presentation. For example, an antibody agent utilized in accordance with the present invention is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (SMIPs™); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

An antibody or antibody agent used in performing the methods of the instant disclosure can be single chained or double chained. In some embodiments, the antibody or antigen binding molecule is single chained. In certain embodiments, the antigen binding molecule is selected from the group consisting of an scFv, a Fab, a Fab′, a Fv, a F(ab′)₂, a dAb, and any combination thereof.

Antibodies and antibody agents include antibody fragments. An antibody fragment comprises a portion of an intact antibody, such as the antigen binding or variable region of the intact antibody. Antibody fragments include, but are not limited to, Fab, Fab′, Fab′-SH, F(ab′)₂, Fv, diabody, single domain antibody, linear antibodies, multispecific formed from antibody fragments antibodies and scFv fragments, and other fragments. Antibodies also include, but are not limited to, polyclonal monoclonal, chimeric dAb (domain antibody), single chain Fab, Fa, F(ab′)₂ fragments, and scFvs. An antibody can be a whole antibody, or immunoglobulin, or an antibody fragment. Antibody fragments can be made by various techniques, including but not limited to proteolytic digestion of an intact antibody as well as production by recombinant host cells (e.g., E. coli, Chinese Hamster Ovary (CHO) cells, or phage), as known in the art.

In some embodiments, an antibody or antibody agent can be a chimeric antibody (see, e.g., U.S. Pat. No. 4,816,567; and Morrison et al., Proc. Natl. Acad. Sci. USA, 81:6851-6855 (1984)). A chimeric antibody can be an antibody in which a portion of the heavy and/or light chain is derived from a particular source or species, while the remainder of the heavy and/or light chain is derived from a different source or species. In one example, a chimeric antibody can comprise a non-human variable region (e.g., a variable region derived from a mouse, rat, hamster, rabbit, or non-human primate, such as a monkey) and a human constant region. In a further example, a chimeric antibody can be a “class switched” antibody in which the class or subclass has been changed from that of the parent antibody. Chimeric antibodies include antigen-binding fragments thereof.

In some embodiments, a chimeric antibody can be a humanized antibody (See, e.g., Almagro and Fransson, Front. Biosci., 13:1619-1633 (2008); Riechmann et al., Nature, 332:323-329 (1988); Queen et al., Proc. Natl Acad. Sci. USA 86:10029-10033 (1989); U.S. Pat. Nos. 5,821,337, 7,527,791, 6,982,321, and 7,087,409; Kashmiri et al., Methods 36:25-34 (2005); Padlan, Mol. Immunol, 28:489-498 (1991); Dall'Acqua et al., Methods, 36:43-60 (2005); Osbourn et al., Methods, 36:61-68 (2005); and Klimka et al., Br. J. Cancer, 83:252-260 (2000)). A humanized antibody is a chimeric antibody comprising amino acid residues from non-human hypervariable regions and amino acid residues from human framework regions. In certain embodiments, a humanized antibody will comprise substantially all of at least one, and typically two, variable domains, in which all or substantially all of the hypervariable regions (e.g., CDRs) correspond to those of a non-human antibody, and all or substantially all of the Framework Regions (FRs) correspond to those of a human antibody. A humanized antibody optionally can comprise at least a portion of an antibody constant region derived from a human antibody.

In some embodiments, an antibody or antibody agent provided herein is a human antibody. Human antibodies can be produced using various techniques known in the art (See, e.g., van Dijk and van de Winkel, Curr. Opin. Pharmacol, 5: 368-74 (2001); and Lonberg, Curr. Opin. Immunol, 20:450-459 (2008)). A human antibody can be one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies may be prepared using methods well known in the art.

### Chimeric Antigen Receptors

As used herein, chimeric antigen receptors (CARs) are proteins that specifically recognize target antigens (e.g., target antigens on cancer cells). When bound to the target antigen, the CAR can activate the immune cell to attack and destroy the cell bearing that antigen (e.g., the cancer cell). CARs can also incorporate costimulatory or signaling domains to increase their potency. See Krause et al., J. Exp. Med., Volume 188, No. 4, 1998 (619-626); Finney et al., Journal of Immunology, 1998, 161: 2791-2797, Song et al., Blood 119:696-706 (2012); Kalos et al., Sci. Transl. Med. 3:95 (2011); Porter et al., N. Engl. J. Med. 365:725-33 (2011), and Gross et al., Annu. Rev. Pharmacol. Toxicol. 56:59-83 (2016); U.S. Pat. Nos. 7,741,465, and 6,319,494.

Chimeric antigen receptors described herein comprise an extracellular domain, a transmembrane domain, and an intracellular domain, wherein the extracellular domain comprises an antigen binding domain that specifically binds to the target.

In some embodiments, antigen-specific CARs further comprise a safety switch and/or one or more monoclonal antibody specific-epitope.

i. Antigen Binding Domains

As discussed above, CARs described herein comprise an antigen binding domain. An “antigen binding domain” as used herein means any polypeptide that binds a specified target antigen. In some embodiments, the antigen binding domain binds to an antigen on a tumor cell. In some embodiments, the antigen binding domain binds to an antigen on a cell involved in a hyperproliferative disease.

In some embodiments, the antigen binding domain comprises a variable heavy chain, variable light chain, and/or one or more CDRs described herein. In some embodiments, the antigen binding domain is a single chain variable fragment (scFv), comprising light chain CDRs CDR1, CDR2 and CDR3, and heavy chain CDRs CDR1, CDR2 and CDR3.

An antigen binding domain is said to be “selective” when it binds to one target more tightly or with higher affinity than it binds to a second target.

The antigen binding domain of the CAR selectively targets a cancer antigen. In some embodiments, the cancer antigen is selected from EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, Muc3, Muc3, Muc16, FAP alpha, Ly6G6D, c6orf23, G6D, MEGT1, NG25, CD19, BCMA, FLT3, CD70, DLL3, CD52 or CD34. In some embodiments, the CAR comprises an antigen binding domain that targets EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, CD52 or CD34.

In some embodiments, the cancer antigen is selected from the group consisting of carbonic anhydrase IX (CAIX), carcinoembryonic antigen (CEA), CDS, CD7, CDIO, CD19, CD20, CD22, CD30, CD33, CD34, CD38, CD41, CD44, CD49f, CD56, CD74, CD123, CD133, CD138, an antigen of a cytomegalovirus (CMV) infected cell (e.g., a cell surface antigen), epithelial glycoprotein (EGP 2), epithelial glycoprotein-40 (EGP-40), epithelial cell adhesion molecule (EpCAM), receptor tyrosine-protein kinases erb-B2,3,4, folate-binding protein (FBP), fetal acetylcholine receptor (AChR), folate receptors, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), κ-light chain, kinase insert domain receptor (KDR), Lewis A (CA19.9), LI cell adhesion molecule (LICAM), melanoma antigen family A, 1 (MAGE-AI), Mucin 16 (Muc-16), Mucin 1 (Muc-1), Mesothelin (MSLN), NKG2D ligands, cancer-testis antigen NY-ESO-1, oncofetal antigen (h5T4), prostate stem cell antigen (PSCA), prostate-specific membrane antigen (PSMA), tumor- associated glycoprotein 72 (TAG-72), vascular endothelial growth factor R2 (VEGF-R2), and Wilms tumor protein (WT-1).

Variants of the antigen binding domains (e.g., variants of the CDRs, VH and/or VL) are also within the scope of the disclosure, e.g., variable light and/or variable heavy chains that each have at least 70-80%, 80-85%, 85-90%, 90-95%, 95-97%, 97-99%, or above 99% identity to the amino acid sequences of antigen binding domain sequences. In some instances, such molecules include at least one heavy chain and one light chain, whereas in other instances the variant forms contain two variable light chains and two variable heavy chains (or subparts thereof). A skilled artisan will be able to determine suitable variants of the antigen binding domains as set forth herein using well-known techniques. In certain embodiments, one skilled in the art can identify suitable areas of the molecule that can be changed without destroying activity by targeting regions not believed to be important for activity.

In some embodiments, the polypeptide structure of the antigen binding domains is based on antibodies, including, but not limited to, monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the antigen binding domain comprises or consists of avimers.

In some embodiments, an antigen binding domain is a scFv. In some embodiments, an antigen-selective CAR comprises a leader or signal peptide.

In other embodiments, the disclosure relates to isolated polynucleotides encoding any one of the antigen binding domains described herein. In some embodiments, the disclosure relates to isolated polynucleotides encoding a CAR. Also provided herein are vectors comprising the polynucleotides, and methods of making same.

In other embodiments, the disclosure relates to isolated polynucleotides encoding any one of the antigen binding domains described herein. In some embodiments, the disclosure relates to isolated polynucleotides encoding a CAR. Also provided herein are vectors comprising the polynucleotides, and methods of making same.

In some embodiments, a CAR-immune cell (e.g., CAR-T cell) which can form a component of a population of cells generated by practicing the methods of the instant disclosure comprises a polynucleotide encoding a safety switch polypeptide, such as for example RQR8 or rituximab mimotope. See, e.g., WO2013153391A, which is hereby incorporated by reference in its entirety. In a CAR-immune cell (e.g., a CAR-T cell) comprising the polynucleotide, the safety switch polypeptide can be expressed at the surface of a CAR-immune cell (e.g., CAR-T cell).

ii. Hinge Domain

The extracellular domain of the CARs of the disclosure can comprise a “hinge” domain (or hinge region). The term generally refers to any polypeptide that functions to link the transmembrane domain in a CAR to the extracellular antigen binding domain in a CAR. In particular, hinge domains can be used to provide more flexibility and accessibility for the extracellular antigen binding domain.

A hinge domain can comprise up to 300 amino acids—in some embodiments 10 to 100 amino acids or in some embodiments 25 to 50 amino acids. The hinge domain can be derived from all or part of naturally occurring molecules, such as from all or part of the extracellular region of CD8, CD4, CD28, 4-1BB, or IgG (in particular, the hinge region of an IgG; it will be appreciated that the hinge region can contain some or all of a member of the immunoglobulin family such as IgG1, IgG2, IgG3, IgG4, IgA, IgD, IgE, IgM, or fragment thereof), or from all or part of an antibody heavy-chain constant region. Alternatively, the hinge domain can be a synthetic sequence that corresponds to a naturally occurring hinge sequence, or can be an entirely synthetic hinge sequence. In some embodiments said hinge domain is a part of human CD8α chain (e.g., NP_001139345.1). In other embodiments, said hinge and transmembrane domains comprise a part of human CD8α chain. In some embodiments, the hinge domain of CARs described herein comprises a subsequence of CD8α, an IgG1, IgG4, PD-1 or an FcγRIIIα, in particular the hinge region of any of an CD8α, an IgG1, IgG4, PD-1 or an FcγRIIIα. In some embodiments, the hinge domain comprises a human CD8α hinge, a human IgG1 hinge, a human IgG4, a human PD-1 or a human FcγRIIIα hinge. In some embodiments the CARs disclosed herein comprise a scFv, CD8a human hinge and transmembrane domains, the CD3ζ signaling domain, and 4-1BB signaling domain.

iii. Transmembrane Domain

The CARs of the disclosure are designed with a transmembrane domain that is fused to the extracellular domain of the CAR. It can similarly be fused to the intracellular domain of the CAR. In some instances, the transmembrane domain can be selected or modified by amino acid substitution to avoid binding of such domains to the transmembrane domains of the same or different surface membrane proteins to minimize interactions with other members of the receptor complex. In some embodiments, short linkers can form linkages between any or some of the extracellular, transmembrane, and intracellular domains of the CAR.

Suitable transmembrane domains for a CAR disclosed herein have the ability to (a) be expressed at the surface an immune cell such as, for example without limitation, a lymphocyte cell, such as a T helper (Tₕ) cell, cytotoxic T (T_{c}) cell, T regulatory (T_{reg}) cell, or Natural killer (NK) cells, and/or (b) interact with the extracellular antigen binding domain and intracellular signaling domain for directing the cellular response of an immune cell against a target cell.

The transmembrane domain can be derived either from a natural or from a synthetic source. Where the source is natural, the domain can be derived from any membrane-bound or transmembrane protein.

Transmembrane regions of particular use in this disclosure can be derived from (comprise, or correspond to) CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1 1d, ITGAE, CD103, ITGAL, CD1 1a, LFA-1, ITGAM, CD1 1b, ITGAX, CD1 1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

As non-limiting examples, the transmembrane region can be derived from, or be a portion of a T cell receptor such as α, β, γ or δ, polypeptide constituting CD3 complex, IL-2 receptor p55 (α chain), p75 (β chain) or γ chain, subunit chain of Fc receptors, in particular Fcγ receptor III or CD proteins. Alternatively, the transmembrane domain can be synthetic and can comprise predominantly hydrophobic residues such as leucine and valine. In some embodiments said transmembrane domain is derived from the human CD8α chain (e.g., NP_001139345.1).

In some embodiments, the transmembrane domain in the CAR of the disclosure is a CD8α transmembrane domain.

In some embodiments, the transmembrane domain in the CAR of the disclosure is a CD28 transmembrane domain.

iv. Intracellular Domain

The intracellular (cytoplasmic) domain of the CARs of the disclosure can provide activation of at least one of the normal effector functions of the immune cell comprising the CAR. Effector function of a T cell, for example, can refer to cytolytic activity or helper activity, including the secretion of cytokines.

In some embodiments, an activating intracellular signaling domain for use in a CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability.

It will be appreciated that suitable (e.g., activating) intracellular domains include, but are not limited to signaling domains derived from (or corresponding to) CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1 1d, ITGAE, CD103, ITGAL, CD1 1a, LFA-1, ITGAM, CD1 1b, ITGAX, CD1 1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

The intracellular domains of the CARs of the disclosure can incorporate, in addition to the activating domains described above, co-stimulatory signaling domains (interchangeably referred to herein as costimulatory molecules) to increase their potency. Costimulatory domains can provide a signal in addition to the primary signal provided by an activating molecule as described herein.

It will be appreciated that suitable costimulatory domains within the scope of the disclosure can be derived from (or correspond to) for example, CD28, OX40, 4-1BB/CD137, CD2, CD3 (alpha, beta, delta, epsilon, gamma, zeta), CD4, CD5, CD7, CD9, CD16, CD22, CD27, CD30, CD 33, CD37, CD40, CD 45, CD64, CD80, CD86, CD134, CD137, CD154, PD-1, ICOS, lymphocyte function-associated antigen-1 (LFA-1 (CD1 1a/CD18), CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNFR, integrin, signaling lymphocytic activation molecule, BTLA, Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1-1d, ITGAE, CD103, ITGAL, CD1-1a, LFA-1, ITGAM, CD1-1b, ITGAX, CD1-1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, or fragments or combinations thereof. It will be appreciated that additional costimulatory molecules, or fragments thereof, not listed above are within the scope of the disclosure.

In some embodiments, the intracellular/cytoplasmic domain of the CAR can be designed to comprise the 4-1BB/CD137 domain by itself or combined with any other desired intracellular domain(s) useful in the context of the CAR of the disclosure. The complete native amino acid sequence of 4-1BB/CD137 is described in NCBI Reference Sequence: NP_001552.2. The complete native 4-1BB/CD137 nucleic acid sequence is described in NCBI Reference Sequence: NM_001561.5.

In some embodiments, the intracellular/cytoplasmic domain of the CAR can be designed to comprise the CD28 domain by itself or combined with any other desired intracellular domain(s) useful in the context of the CAR of the disclosure. The complete native amino acid sequence of CD28 is described in NCBI Reference Sequence: NP_006130.1. The complete native CD28 nucleic acid sequence is described in NCBI Reference Sequence: NM_006139.1.

In some embodiments, the intracellular/cytoplasmic domain of the CAR can be designed to comprise the CD3 zeta domain by itself or combined with any other desired intracellular domain(s) useful in the context of the CAR of the disclosure.

For example, the intracellular domain of the CAR can comprise a CD3 zeta chain portion and a portion of a costimulatory signaling molecule. The intracellular signaling sequences within the intracellular signaling portion of the CAR of the disclosure can be linked to each other in a random or specified order. In some embodiments, the intracellular domain is designed to comprise the activating domain of CD3 zeta and a signaling domain of CD28. In some embodiments, the intracellular domain is designed to comprise the activating domain of CD3 zeta and a signaling domain of 4-1BB.

In some embodiments the intracellular signaling domain of the CAR of the disclosure comprises a domain of a co-stimulatory molecule. In some embodiments, the intracellular signaling domain of a CAR of the disclosure comprises a part of co-stimulatory molecule selected from the group consisting of fragment of 4-1BB (GenBank: AAA53133.) and CD28 (NP_006130.1).

### Genetic Modifications of CAR T Cells

Also provided herein are engineered immune cells and populations of engineered immune cells expressing CAR (e.g., CAR-T cells or CAR-cells), which are depleted of cells expressing endogenous TCR.

In some embodiments, an engineered immune cell comprises a CAR T cell, each CAR T cell comprising an extracellular antigen-binding domain and has reduced or eliminated expression of endogenous TCR. In some embodiments, a population of engineered immune cells comprises a population of CAR T cells, each CAR T cell comprising two or more different extracellular antigen-binding domain and has reduced or eliminated expression of endogenous TCR. In some embodiments, an immune cell comprises a population of CARs, each CAR T cell comprising the same extracellular antigen-binding domains and has reduced or eliminated expression of endogenous TCR.

In some embodiments, an engineered immune cell according to the present disclosure comprises one disrupted or inactivated gene selected from the group consisting of CD52, DLL3, GR, PD-1, CTLA-4, LAG3, TIM3, BTLA, BY55, TIGIT, B7H5, LAIR1, SIGLEC10, 2B4, HLA, TCRα and TCRβ and/or expresses a CAR, a multi-chain CAR and/or a pTaα transgene. In some embodiments, an isolated cell comprises polynucleotides encoding polypeptides comprising a multi-chain CAR. In some embodiments, the isolated cell according to the present disclosure comprises two disrupted or inactivated genes selected from the group consisting of: CD52 and GR, CD52 and TCRα, CDR52 and TCRβ, DLL3 and CD52, DLL3 and TCRα, DLL3 and TCRβ, GR and TCRα, GR and TCRβ, TCRα and TCRβ, PD-1 and TCRα, PD-1 and TCRβ, CTLA-4 and TCRα, CTLA-4 and TCRβ, LAG3 and TCRβ, LAG3 and TCRβ, TIM3and TCRα, Tim3 and TCRβ, BTLA and TCRβ, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ and/or expresses a CAR, including a multi-chain CAR, and/or a pTα transgene. In some embodiments the method comprises disrupting or inactivating one or more genes by introducing into the cells an endonuclease capable of selectively inactivating a gene by selective DNA cleavage. In some embodiments the endonuclease can be, for example, a zinc finger nuclease (ZFN), megaTAL nuclease, meganuclease, transcription activator-like effector nuclease (TALE-nuclease, or TALEN®), or CRISPR (e.g., Cas9 or Cas12) endonuclease.

In some embodiments, TCR is rendered not functional in the cells according to the disclosure by disrupting or inactivating TCRα gene and/or TCRβ gene(s). In some embodiments, TCR is rendered not functional in the cells according to the disclosure by disrupting or inactivating the TCRα constant region (the TRAC locus). In some embodiments, a method to obtain modified cells derived from an individual is provided, wherein the cells can proliferate independently of the major histocompatibility complex (MHC) signaling pathway. Modified cells, which can proliferate independently of the MHC signaling pathway, susceptible to be obtained by this method are encompassed in the scope of the present disclosure. Modified cells disclosed herein can be used for treating patients in need thereof against Host versus Graft (HvG) rejection and Graft versus Host Disease (GvHD); therefore in the scope of the present disclosure is a method of treating patients in need thereof against Host versus Graft (HvG) rejection and Graft versus Host Disease (GvHD) comprising treating said patient by administering to said patient an effective amount of modified cells comprising disrupted or inactivated TCRα and/or TCRβ genes.

The present disclosure provides methods of determining the purity of a population of engineered immune cells lacking or having reduced endogenous TCR expression. In some embodiments, the engineered immune cells comprise less than 5.0%, less than 4.0%, less than 3.0% TCR+ cells, less than 2.0% TCR+ cells, less than 1.0% TCR+ cells, less than 0.9% TCR+ cells, less than 0.8% TCR+ cells, less than 0.7% TCR+ cells, less than 0.6% TCR+ cells, less than 0.5% TCR+ cells, less than 0.4% TCR+ cells, less than 0.3% TCR+ cells, less than 0.2% TCR+ cells, or less than 0.1% TCR+ cells. Such a population can be a product of the disclosed methods.

In some embodiments, an engineered immune cell according to the present disclosure can comprise one or more disrupted or inactivated genes. In some embodiments, a gene for a target antigen (e.g., EGFRvIII, Flt3, WT-1, CD20, CD23, CD30, CD38, CD33, CD133, MHC-WT1, TSPAN10, WHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, or CD34, CD70) can be knocked out to introduce a CAR targeting the same antigen (e.g., a EGFRvIII, Flt3, WT-1, CD20, CD23, CD30, CD38, CD33, CD133, TSPAN10, MHC-PRAME, Liv1, ADAM10, CHRNA2, LeY, NKGD2D, CS1, CD44v6, ROR1 , Claudin-18.2, Muc17, FAP alpha, Ly6G6D, c6orf23, G6D, MEGT1, NG25, CD19, BCMA, FLT3, CD70, DLL3, or CD34, CD70 CAR) to avoid induced CAR activation. As described herein, in some embodiments, an engineered immune cell according to the present disclosure comprises one disrupted or inactivated gene selected from the group consisting of MHC1 (β2M), MHC2 (CIITA), EGFRvIII, Flt3, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, or CD34, CD70, TCRα and TCRβ and/or expresses a CAR or a multi-chain CAR. In some embodiments, a cell comprises a multi-chain CAR. In some embodiments, the isolated cell comprises two disrupted or inactivated genes selected from the group consisting of: CD52 and TCRα, CDR52 and TCRβ, PD-1 and TCRα, PD-1 and TCRβ, MHC-1 and TCRα, MHC-1 and TCRβ, MHC2 and TCRα, MHC2 and TCRβ and/or expresses a CAR or a multi-chain CAR.

The engineered immune cells can be allogeneic or autologous.

In some embodiments, an engineered immune cell or population of engineered immune cells comprises a T cell (e.g., inflammatory T-lymphocyte, cytotoxic T-lymphocyte, regulatory T-lymphocyte, helper T-lymphocyte, tumor infiltrating lymphocyte (TIL)), NK cell, NK-T-cell, TCR-expressing cell, dendritic cell, killer dendritic cell, a mast cell, or a B-cell, and expresses a CAR. In some embodiments, the T cell can be derived from the group consisting of CD4+ T lymphocytes, CD8+ T lymphocytes or population comprising a combination of CD4+ and CD8+ T cells.

In some embodiments, an engineered immune cell or population of engineered immune cells that are generated using the disclosed methods can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells.

In some embodiments, an engineered immune cell or a population of immune cells that are generated using the disclosed methods is obtained or prepared from peripheral blood. In some embodiments, an engineered immune cell is obtained or prepared from peripheral blood mononuclear cells (PBMCs). In some embodiments, an engineered immune cell is obtained or prepared from bone marrow. In some embodiments, an engineered immune cell is obtained or prepared from umbilical cord blood. In some embodiments, the cell is a human cell. In some embodiments, the cell is transfected or transduced by the nucleic acid vector using a method selected from the group consisting of electroporation, sonoporation, biolistics (e.g., Gene Gun), lipid transfection, polymer transfection, nanoparticles, viral transfection (e.g., retrovirus, lentivirus, AAV) or polyplexes.

In some embodiments, the engineered immune cells expressing at their cell surface membrane an antigen-specific CAR comprise a percentage of stem cell memory and central memory cells greater than 10%, 20%, 30%, 40%, 50%, 60%, 70%, 80%, 90%, or 100%.

In some embodiments, engineered immune cells expressing at their cell surface membrane an antigen-specific CAR comprise a percentage of stem cell memory and central memory cells of about 10% to about 60%, about 10% to about 50%, about 10% to about 40%, about 15% to about 50%, about 15% to about 40%, about 20% to about 60%, or about 20% to about 70%.

In some embodiments, engineered immune cells expressing at their cell surface membrane an antigen-specific CAR enriched in T_{CM} and/or T_{SCM} cells such that the engineered immune cells comprise at least about 60%, 65%, 70%, 75%, or 80% combined T_{CM} and T_{SCM} cells. In some embodiments, engineered immune cells expressing at their cell surface membrane an antigen-specific CAR are enriched in T_{CM} and/or T_{SCM} cells such that the engineered immune cells comprise at least about 70% combined T_{CM} and T_{SCM} cells. In some embodiments, engineered immune cells expressing at their cell surface membrane an antigen-specific CAR e enriched in T_{CM} and/or T_{SCM} cells such that the engineered immune cells comprise at least about 75% combined T_{CM} and/or T_{SCM} cells.

In some embodiments, an engineered immune cell is an inflammatory T-lymphocyte that expresses a CAR. In some embodiments, an engineered immune cell is a cytotoxic T-lymphocyte that expresses a CAR. In some embodiments, an engineered immune cell is a regulatory T-lymphocyte that expresses a CAR. In some embodiments, an engineered immune cell is a helper T-lymphocyte that expresses a CAR.

In some embodiments, the immune cells are engineered to be resistant to one or more chemotherapy drugs. The chemotherapy drug can be, for example, a purine nucleotide analogue (PNA), thus making the immune cell suitable for cancer treatment combining adoptive immunotherapy and chemotherapy. Exemplary PNAs include, for example, clofarabine, fludarabine, cyclophosphamide, and cytarabine, alone or in combination. PNAs are metabolized by deoxycytidine kinase (dCK) into mono-, di-, and tri-phosphate PNA. Their tri-phosphate forms compete with ATP for DNA synthesis, act as pro-apoptotic agents, and are potent inhibitors of ribonucleotide reductase (RNR), which is involved in trinucleotide production.

In some embodiments, isolated cells or cell lines of the disclosure can comprise a pTα or a functional variant thereof. In some embodiments, an isolated cell or cell line can be further genetically modified by disrupting or inactivating the TCRα gene.

The disclosure also provides engineered immune cells comprising any of the CAR polynucleotides described herein. In some embodiments, a CAR can be introduced into an immune cell as a transgene via a plasmid vector. In some embodiments, the plasmid vector can also contain, for example, a selection marker which provides for identification and/or selection of cells which received the vector.

CAR polypeptides can be synthesized in situ in the cell after introduction of polynucleotides encoding the CAR polypeptides into the cell. Alternatively, CAR polypeptides can be produced outside of cells, and then introduced into cells. Methods for introducing a polynucleotide construct into cells are known in the art. In some embodiments, stable transformation methods (e.g., using a lentiviral vector) can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides can be introduced into a cell by any suitable means such as for example, recombinant viral vectors (e.g., retroviruses, adenoviruses), liposomes, and the like. Transient transformation methods include, for example without limitation, transduction, microinjection, electroporation or particle bombardment. Polynucleotides can be included in vectors, such as for example plasmid vectors or viral vectors.

In some embodiments, isolated nucleic acids are provided comprising a promoter operably linked to a first polynucleotide encoding an antigen binding domain, at least one costimulatory molecule, and an activating domain. In some embodiments, the nucleic acid construct is contained within a viral vector. In some embodiments, the viral vector is selected from the group consisting of retroviral vectors, murine leukemia virus vectors, SFG vectors, adenoviral vectors, lentiviral vectors, adeno-associated virus (AAV) vectors, Herpes virus vectors, and vaccinia virus vectors. In some embodiments, the nucleic acid is contained within a plasmid.

In some embodiments, the isolated nucleic construct is contained within a viral vector and is introduced into the genome of an engineered immune cell by random integration, e.g., lentiviral- or retroviral-mediated random integration. In some embodiments, the isolated nucleic acid construct is contained in a viral vector or a non-viral vector and is introduced into the genome of an engineered immune cell by site-specific integration by homologous recombination, e.g., adenovirus-mediated site-specific integration.

### Manufacture of Engineered Immune Cells

A variety of known techniques can be utilized in making the polynucleotides, polypeptides, vectors, antigen binding domains, immune cells, compositions, and the like according to the disclosure.

Prior to the in vitro manipulation or genetic modification of the immune cells described herein, the cells can be obtained from a subject. Cells expressing a CAR can be derived from an allogeneic or autologous source and can be depleted of endogenous TCR as described herein.

a. Source Material

In some embodiments, the immune cells comprise T cells. T cells can obtained from a number of sources, including peripheral blood mononuclear cells (PBMCs), bone marrow, lymph nodes tissue, cord blood, thymus tissue, tissue from a site of infection, ascites, pleural effusion, spleen tissue, and tumors. In some embodiments, T cells can be obtained from a volume of blood collected from the subject using any number of techniques known to the skilled person, such as FICOLL™ separation.

Cells can be obtained from the circulating blood of an individual by apheresis. The apheresis product typically contains lymphocytes, including T cells, monocytes, granulocytes, B cells, other nucleated white blood cells, red blood cells, and platelets. In some embodiments, the cells collected by apheresis can be washed to remove the plasma fraction, and then placed in an appropriate buffer or media for subsequent processing.

In some embodiments, T cells are isolated from PBMCs by lysing the red blood cells and depleting the monocytes, for example, using centrifugation through a PERCOLL™ gradient. A specific subpopulation of T cells, (e.g., CD28+, CD4+, CD45RA−, and CD45RO+T cells or CD28+, CD4+, CDS+, CD45RA−, CD45RO+, and CD62L+ T cells) can be further isolated by positive or negative selection techniques known in the art. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. Flow cytometry and cell sorting can also be used to isolate cell populations of interest for use in the present disclosure.

PBMCs can be used directly for genetic modification with the immune cells (such as CARs or TCRs) using methods as described herein. In certain embodiments, after isolating the PBMCs, T lymphocytes can be further isolated and both cytotoxic and helper T lymphocytes can be sorted into naive, memory, and effector T cell subpopulations either before or after genetic modification and/or expansion. In some embodiments, CD8+ cells are further sorted into naive, stem cell memory, central memory, and effector cells by identifying cell surface antigens that are associated with each of these types of CD8+ cells. In some embodiments, the expression of phenotypic markers of central memory T cells include CD27, CD45RA, CD45RO, CD62L, CCR7, CD28, CD3, and CD127 and are negative for granzyme B. In some embodiments, stem cell memory T cells are CD45RO−, CD62L+, CD8+ T cells. In some embodiments, central memory T cells are CD45RO+, CD62L+, CD8+ T cells. In some embodiments, effector T cells are negative for CD62L, CCR7, CD28, and CD127, and positive for granzyme B and perforin. In some embodiments, CD4+ T cells are further sorted into subpopulations. For example, CD4+ T helper cells can be sorted into naive, central memory, and effector cells by identifying cell populations that have cell surface antigens.

b. Stem Cell Derived Immune Cells

In some embodiments, the immune cells can be derived from embryonic stem (ES) or induced pluripotent stem (iPS) cells. Suitable HSCs, mesenchymal, iPS cells and other types of stem cells can be cultivated immortal cell lines or isolated directly from a patient. Various methods for isolating, developing, and/or cultivating stem cells are known in the art and can be used to practice the present disclosure.

In some embodiments, the immune cell is an induced pluripotent stem cell (iPSC) derived from a reprogrammed T-cell. In some embodiments, the source material can be an induced pluripotent stem cell (iPSC) derived from a T cell or a non-T cell. In some embodiments, the immune cell is an iPSC-derived T cell. In some embodiments, the immune cell is an iPSC-derived NK cells. The source material can be an embryonic stem cell. The source material can be a B cell, or any other cell from peripheral blood mononuclear cell isolates, hematopoietic progenitor, hematopoietic stem cell, mesenchymal stem cell, adipose stem cell, or any other somatic cell type.

c. Genetic Modification of Isolated Cells

The immune cells, such as T cells, can be genetically modified following isolation using known methods, or the immune cells can be activated and expanded (or differentiated in the case of progenitors) in vitro prior to being genetically modified. In some embodiments, the isolated immune cells can be activated by contacting the cells with a volume of an anti-CD3/CD28 nanomatrix, for example, TransAct™, before being genetically modified.

In some embodiments, the isolated immune cells are genetically modified to reduce or eliminate expression of endogenous TCRα and/or CD52. In some embodiments, the cells are genetically modified using gene editing technology (e.g., CRISPR/Cas9, CRISPR/Cas12a, a zinc finger nuclease (ZFN), a TALEN, a MegaTAL, a meganuclease) to reduce or eliminate expression of endogenous proteins (e.g., TCRα and/or CD52). In another embodiment, the immune cells, such as T cells, are genetically modified with the chimeric antigen receptors described herein (e.g., transduced with a viral vector comprising one or more nucleotide sequences encoding a CAR) and then are activated and/or expanded in vitro.

Certain methods for making the constructs and engineered immune cells of the disclosure are described in PCT application PCT/US15/14520, the contents of which are hereby incorporated by reference in their entirety.

It will be appreciated that PBMCs can further include other cytotoxic lymphocytes such as NK cells or NKT cells. An expression vector carrying the coding sequence of a chimeric receptor as disclosed herein can be introduced into a population of human donor T cells, NK cells or NKT cells. Successfully transduced T cells that carry the expression vector can be sorted using flow cytometry to isolate CD3 positive T cells and then further propagated to increase the number of these CAR expressing T cells in addition to cell activation using anti-CD3 antibodies and IL-2 or other methods known in the art as described elsewhere herein. Standard procedures are used for cryopreservation of T cells expressing the CAR for storage and/or preparation for use in a human subject. In one embodiment, the in vitro transduction, culture and/or expansion of T cells are performed in the absence of non-human animal derived products such as fetal calf serum and fetal bovine serum.

For cloning of polynucleotides, the vector can be introduced into a host cell (an isolated host cell) to allow replication of the vector itself and thereby amplify the copies of the polynucleotide contained therein. The cloning vectors can contain sequence components generally include, without limitation, an origin of replication, promoter sequences, transcription initiation sequences, enhancer sequences, and selectable markers. These elements can be selected as appropriate by a person of ordinary skill in the art. For example, the origin of replication can be selected to promote autonomous replication of the vector in the host cell.

In some embodiments, the present disclosure provides isolated host cells containing the vector provided herein. The host cells containing the vector can be useful in expression or cloning of the polynucleotide contained in the vector. Suitable host cells can include, without limitation, prokaryotic cells, fungal cells, yeast cells, or higher eukaryotic cells such as mammalian cells, particularly human cells.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art. In a further embodiment, a mixture of different expression vectors can be used in genetically modifying a donor population of immune effector cells wherein each vector encodes a different CAR as disclosed herein. The resulting transduced immune effector cells form a mixed population of engineered cells, with a proportion of the engineered cells expressing more than one different CARs.

In one embodiment, the disclosure provides a method of storing genetically engineered cells expressing CARs or TCRs. This involves cryopreserving the immune cells such that the cells remain viable upon thawing. A fraction of the immune cells expressing the CARs can be cryopreserved by methods known in the art to provide a permanent source of such cells for the future treatment of patients afflicted with a malignancy. When needed, the cryopreserved transformed immune cells can be thawed, grown and expanded for more such cells.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer's lactate can be utilized. The infusion medium can be supplemented with human serum albumin.

d. Allogeneic CAR T Cells

The process for manufacturing allogeneic CAR T therapy involves harvesting peripheral blood mononuclear cells (PBMCs) from healthy, selected, screened and tested donors. Next, the cells are engineered to express CARs, which recognize certain cell surface proteins that are expressed in hematologic or solid tumors. Allogeneic T cells are gene editing to reduce the risk of graft versus host disease (GvHD) and to prevent allogeneic rejection. A T cell receptor gene (e.g., TCRα, TCRβ) is knocked out to avoid GvHD. The CD52 gene can be knocked out to render the CAR T product resistant to anti-CD52 antibody treatment. Anti-CD52 antibody treatment can therefore be used to suppress the host immune system and allow the CAR T to stay engrafted to achieve full therapeutic impact. The engineered T cells then undergo a purification step and are ultimately cryopreserved in vials for delivery to patients.

e. Autologous CAR T Cells

Autologous chimeric antigen receptor (CAR) T cell therapy involves collecting a patient's own cells (e.g., white blood cells, including T cells) and genetically engineering the T cells to express CARs that recognize target expressed on the cell surface of one or more specific cancer cells and kill cancer cells. The engineered cells are then cryopreserved and subsequently administered to the patient.

### Control of Adventitious Viral Infection

HHV-6 can infect immune cells, including T cells, and establish latent infection (or latency). Viral reactivation may be observed, though in rare events, in in vitro cell culture over a period of time. As disclosed herein, one or more exogenous cell culture additives or agents can be added during the cell culture of manufacturing process of cell-based therapies to prevent, control, or inhibit viral replication, or prevent, control, or inhibit viral spreading, after reactivation of any endemic latent viruses present in the source immune cells that are obtained from, e.g., a patient or a healthy individual. In some embodiments, the source immune cells are T cells, PBMCs, NK cells, or iPSCs. In some embodiments, the exogenous agent can be IFN. In some embodiments, the viral replication is HHV-6 viral replication. In some embodiments, the endemic latent virus is HHV-6.

In some embodiments, the exogenous agent is a type I IFN of a type III IFN. In certain embodiments, the IFN is IFNα or IFNβ. In some embodiments, the IFN is added to the culture medium at a concentration of about 0.01 ng/ml to about 100 ng/ml, about 0.01 ng/ml to about 10 ng/ml, about 0.01 ng/ml to about 1 ng/ml, about 0.01 ng/ml to about 0.1 ng/ml, about 0.1 ng/ml to about 100 ng/ml, about 0.1 ng/ml to about 10 ng/ml, about 0.1 ng/ml to about 1 ng/ml, about 0.1 ng/ml to about 0.9 ng/ml, about 0.1 ng/ml to about 0.8 ng/ml, about 0.1 ng/ml to about 0.7 ng/ml, about 0.1 ng/ml to about 0.6 ng/ml, or about 0.1 ng/ml to about 0.5 ng/ml, about 0.1 ng/ml, about 0.2 ng/ml, about 0.3 ng/ml, about 0.4 ng/ml, about 0.5 ng/ml, about 0.6 ng/ml, about 0.7 ng/ml. about 0.8 ng/ml, about 0.9 ng/ml, or about 1 ng/ml. In certain embodiments, the IFN is human IFNα. An exemplary human IFNα sequence is shown below (NCBI Accession No. NP_000596.2, IFNα 2a):

MALTFALLVA LLVLSCKSSC SVGCDLPQTH SLGSRRTLML
LAQMRRISLF SCLKDRHDFG FPQEEFGNQF QKAETIPVLH
EMIQQIFNLF STKDSSAAWD ETLLDKFYTE LYQQLNDLEA
CVIQGVGVTE TPLMKEDSIL AVRKYFQRIT LYLKEKKYSP
CAWEVVRAEI MRSFSLSTNL QESLRSKE (SEQ ID NO: 19,
the underlined indicates signal peptide)

An exemplary human IFNα sequence without the signal peptide is shown below:

(SEQ ID NO: 20)
CDLPQTH SLGSRRTLML LAQMRRISLF SCLKDRHDFG
FPQEEFGNQF QKAETIPVLH EMIQQIFNLF STKDSSAAWD
ETLLDKFYTE LYQQLNDLEA CVIQGVGVTE TPLMKEDSIL
AVRKYFQRIT LYLKEKKYSP CAWEVVRAEI MRSFSLSTNL
QESLRSKE

In some embodiments, the IFNα comprises the amino acid sequence having at least 95%, at least 96%, at least 97%, at least 98%, at least 99% or 100% sequence identity to SEQ ID NO: 19 with or without the signal peptide. In some embodiments, the IFNα comprises the amino acid sequence having at least 95%, at least 96%, at least 97%, at least 98%, at least 99% or 100% sequence identity to SEQ ID NO: 20.

In some embodiments, the antiviral agent is foscarnet, or a salt thereof. In some embodiments, the foscarnet, or a salt, e.g., sodium salt, thereof, is added to the culture medium at a concentration of about 8 μM to about 20 μM, about 8 μM to about 15 μM, about 8 μM, about 9 μM, about 10 μM, about 11 μM, about 12 μM, about 13 μM, about 14 μM, or about 15 μM. In some embodiments, the antiviral is ganciclovir, or a salt thereof. In some embodiments, the ganciclovir, or a salt thereof, is added to the culture medium at a concentration of about 25 μM to about 35 μM, about 25 μM to about 30 μM, about 27 μM to about 30 μM, about 25 μM, about 26 μM, about 27 μM, about 28 μM, about 29 μM, about 30 μM, or about 35 μM. In some embodiments, the antiviral is cidofovir, or a salt thereof. In some embodiments, the cidofovir, or a salt thereof, is added to the culture medium at a concentration of about 14 μM to about 25 μM, about 14 μM to about 20 μM, about 14 μM, about 15 μM, about 16 μM, about 17 μM, about 18 μM, about 19 μM, or about 20 μM.

The structure of foscarnet is shown below (sodium salt CAS No. 63585-09-1):

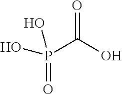

The structure of ganciclovir is shown below (CAS No. 82410-32-0):

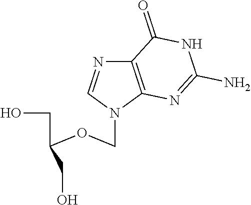

The structure of cidofovir is shown below (CAS No. 113852-37-2):

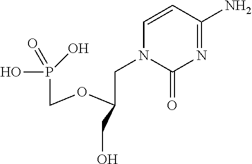 [SMILES: Nc1ccn(C[C@@H](CO)OCP(=O)(O)O)c(=O)n1]

The one or more exogenous additives or agents can be added to the culture medium at different time points of cell culture in the manufacturing process. In some embodiments, the additive is added on day 0, day 1, day 2, day 3, day 4, day 5, day 6, day 7, day 8, day 9, day 10, day 11, day 12, day 13, day 14, day 15, day 16, day 17, day 18, day 19, day 20, or day 21 or beyond day 21 of the manufacturing process. In some embodiments, the additive is added from day 1 to day 5, day 6 to day 10, day 10 to day 15, or day 15 to day 20, or after day 20 of the manufacturing process. In some embodiments, the additive is added from day 5 to day 10, day 6 to day 10, day 7 to day 10, day 8 to day 10, day 9 to day 10, day 6 to day 11, day 7 to day 11, day 8 to day 11, day 9 to day 11, day 10 to day 11, day 5 to day 9, day 6 to day 9, day 7 to day 9, or day 8 to day 9 of the manufacturing process. In some embodiments, the additive is added from day 9 to day 15, day 10 to day 15, day 11 to day 15, day 12 to day 15, day 13 to day 15, day 14 to day 15, day 9 to day 13, day 9 to day 12, day 9 to day 11, day 9 to day 10, day 10 to day 13, day 10 to day 14, or day 10 to day 15 of the manufacturing process. In some embodiments, the additive is added from day 6 to day 11, day 7 to day 11, or day 8 to day 11 of the manufacturing process. In some embodiments, the cells are in contact with IFN in the culture medium for about 1 day, about 2 days, about 3 days, about 4 days, about 5 days, about 6 days, about 7 days, about 8 days, about 9 days or about 10 days.

### Pharmaceutical Composition

In some embodiments, the cells are formulated by first harvesting them from their culture medium, and then washing and concentrating the cells in a medium and container system suitable for administration (with a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer's lactate can be utilized. The infusion medium can be supplemented with human serum albumin.

In embodiments, desired treatment amounts of cells in the composition are generally at least 2 cells (for example, at least 1 CD8+ central or stem cell memory T cell and at least 1 CD4+ helper T cell subset; or two or more CD8+ central or stem cell memory T cell; or two or more CD4+ helper T cell subset) or is more typically greater than 10² cells, and up to and including 10⁶ up to and including 10⁷, 10⁸ or 10⁹ cells and can be more than 10¹⁰ cells. The number of cells will depend upon the desired use for which the composition is intended, and the type of cells included therein. The density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present disclosure, particularly since all the infused cells will be redirected to a particular target antigen, lower numbers of cells, in the range of about 10⁵/kilogram or about 10⁶/kilogram (10⁶-10¹¹ per patient) can be administered. CAR treatments can be administered multiple times at dosages within these ranges. The cells can be autologous, allogeneic, or heterologous to the patient undergoing therapy.

The CAR expressing cell populations of the present disclosure can be administered either alone, or as a pharmaceutical composition in combination with diluents and/or with other components such as IL-2 or other cytokines or cell populations. Pharmaceutical compositions of the present disclosure can comprise a CAR or TCR expressing cell population, such as T cells, as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

The pharmaceutical compositions (solutions, suspensions or the like), can include one or more of the following: sterile diluents such as water for injection, saline solution, preferably physiological saline, Ringer's solution, isotonic sodium chloride, fixed oils such as synthetic mono- or diglycerides which can serve as the solvent or suspending medium, polyethylene glycols, glycerin, propylene glycol or other solvents; antibacterial agents such as benzyl alcohol or methyl paraben; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic. An injectable pharmaceutical composition is preferably sterile.

### Methods of Treatment

The disclosure comprises methods for treating or preventing a disease (e.g., cancer) in a patient, comprising administering to a patient in need thereof an effective amount of CART cells, or engineered immune cells comprising a CAR disclosed herein. In some embodiments, the effective amount of CAR T cells or engineered immune cells have been analyzed for various attributes according to the methods described in the instant disclosure. In some embodiments, the CAR T cell drug product for therapeutic use has been analyzed for various attributes, such as potency or polyfunctionality according to the methods described in the instant disclosure. In some embodiments, the CAR T cells are TCR− CAR T cells, and the CAR T drug product for therapeutic use has been analyzed for various attributes, such as the amount or percentage of remaining TCR+ CAR T cells and/or potency or polyfunctionality according to the methods described in the instant disclosure.

Methods are provided for treating diseases or disorders, including cancer. In some embodiments, the disclosure relates to creating a T cell-mediated immune response in a subject, comprising administering an effective amount of the engineered immune cells of the present application to the subject. In some embodiments, the T cell-mediated immune response is directed against a target cell or cells. In some embodiments, the engineered immune cell comprises a chimeric antigen receptor (CAR). In some embodiments, the target cell is a tumor cell. In some aspects, the disclosure comprises a method for treating or preventing a malignancy, said method comprising administering to a subject in need thereof an effective amount of at least one isolated antigen binding domain described herein. In some aspects, the disclosure comprises a method for treating or preventing a malignancy, said method comprising administering to a subject in need thereof an effective amount of at least one immune cell, wherein the immune cell comprises at least one chimeric antigen receptor, T cell receptor, and/or isolated antigen binding domain as described herein. The CAR containing immune cells of the disclosure can be used to treat malignancies involving aberrant expression of biomarkers. In some embodiments, CAR containing immune cells of the disclosure can be used to treat small cell lung cancer, melanoma, low grade gliomas, glioblastoma, medullary thyroid cancer, carcinoids, dispersed neuroendocrine tumors in the pancreas, bladder and prostate, testicular cancer, and lung adenocarcinomas with neuroendocrine features. In exemplary embodiments, the CAR containing immune cells, e.g., CAR-T cells of the disclosure are used to treat small cell lung cancer.

Also provided are methods for reducing the size of a tumor in a subject, comprising administering to the subject an engineered cell of the present disclosure to the subject, wherein the cell comprises a chimeric antigen receptor comprising an antigen binding domain and binds to an antigen on the tumor.

In some embodiments, the subject has a solid tumor, or a blood malignancy such as lymphoma or leukemia. In some embodiments, the engineered cell is delivered to a tumor bed. In some embodiments, the cancer is present in the bone marrow of the subject. In some embodiments, the engineered cells are autologous immune cells, e.g., autologous T cells. In some embodiments, the engineered cells are allogeneic immune cells, e.g., allogeneic T cells. In some embodiments, the engineered cells are heterologous immune cells, e.g., heterologous T cells. In some embodiments, the engineered cells of the present application are transfected or transduced in vivo. In other embodiments, the engineered cells are transfected or transduced ex vivo. As used herein, the term “in vitro cell” refers to any cell which is cultured ex vivo.

A “therapeutically effective amount,” “effective dose,” “effective amount,” or “therapeutically effective dosage” of a therapeutic agent, e.g., engineered CART cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “patient” and “subject” are used interchangeably and include human and non-human animal subjects as well as those with formally diagnosed disorders, those without formally recognized disorders, those receiving medical attention, those at risk of developing the disorders, etc.

The term “treat” and “treatment” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. The term “prevent” does not require the 100% elimination of the possibility of an event. Rather, it denotes that the likelihood of the occurrence of the event has been reduced in the presence of the compound or method.

Desired treatment amounts of cells in the composition is generally at least 2 cells (for example, at least 1 CD8+ central memory T cell and at least 1 CD4+ helper T cell subset) or is more typically greater than 10² cells, and up to 10⁶, up to and including 10⁸ or 10⁹ cells and can be more than 10¹⁰ cells. The number of cells will depend upon the desired use for which the composition is intended, and the type of cells included therein. The density of the desired cells is typically greater than 10⁶ cells/ml and generally is greater than 10⁷ cells/ml, generally 108 cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present disclosure, particularly since all the infused cells will be redirected to a particular target antigen, lower numbers of cells, in the range of 10⁶/kilogram (10⁶-10¹¹ per patient) can be administered. CAR treatments can be administered multiple times at dosages within these ranges. The cells can be autologous, allogeneic, or heterologous to the patient undergoing therapy.

In some embodiments, the therapeutically effective amount of the CAR T cells is about 1×10⁵ cells/kg, about 2×10⁵ cells/kg, about 3×10⁵ cells/kg, about 4×10⁵ cells/kg, about 5×10⁵ cells/kg, about 6×10⁵ cells/kg, about 7×10⁵ cells/kg, about 8×10⁵ cells/kg, about 9×10⁵ cells/kg, 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

In some embodiments, target doses for CAR+/CAR-T+/TCR+ cells range from 1×10⁶-2×10⁸ cells/kg, for example 2×10⁶ cells/kg. It will be appreciated that doses above and below this range can be appropriate for certain subjects, and appropriate dose levels can be determined by the healthcare provider as needed. Additionally, multiple doses of cells can be provided in accordance with the disclosure.

In some aspect, the disclosure comprises a pharmaceutical composition comprising at least one antigen binding domain as described herein and a pharmaceutically acceptable excipient. In some embodiments, the pharmaceutical composition further comprises an additional active agent.

The CAR expressing cell populations of the present disclosure can be administered either alone, or as a pharmaceutical composition in combination with diluents and/or with other components such as IL-2 or other cytokines or cell populations. Pharmaceutical compositions of the present disclosure can comprise a CAR or TCR expressing cell population, such as T cells, as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

The pharmaceutical compositions (solutions, suspensions or the like), can include one or more of the following: sterile diluents such as water for injection, saline solution, preferably physiological saline, Ringer's solution, isotonic sodium chloride, fixed oils such as synthetic mono- or diglycerides which can serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic. An injectable pharmaceutical composition is preferably sterile.

In some embodiments, upon administration to a patient, engineered immune cells expressing at their cell surface any one of the antigen-specific CARs described herein can reduce, kill or lyse endogenous antigen-expressing cells of the patient. In one embodiment, a percentage reduction or lysis of antigen-expressing endogenous cells or cells of a cell line expressing an antigen by engineered immune cells expressing any one of an antigen-specific CARs described herein is at least about or greater than 10%, 15%, 20%, 25%, 30%, 35%, 40%, 45%, 50%, 55%, 60%, 65%, 70%, 75%, 80%, 85%, 90%, or 95%. In one embodiment, a percentage reduction or lysis of antigen-expressing endogenous cells or cells of a cell line expressing an antigen by engineered immune cells expressing antigen-specific CARs is about 5% to about 95%, about 10% to about 95%, about 10% to about 90%, about 10% to about 80%, about 10% to about 70%, about 10% to about 60%, about 10% to about 50%, about 10% to about 40%, about 20% to about 90%, about 20% to about 80%, about 20% to about 70%, about 20% to about 60%, about 20% to about 50%, about 25% to about 75%, or about 25% to about 60%. In one embodiment, the endogenous antigen-expressing cells are endogenous antigen-expressing bone marrow cells.

In one embodiment, the percent reduction or lysis of target cells, e.g., a cell line expressing an antigen, by engineered immune cells expressing at their cell surface membrane an antigen-specific CAR of the disclosure can be measured using the assay disclosed herein.

The methods can further comprise administering one or more chemotherapeutic agent. In some embodiments, the chemotherapeutic agent is a lymphodepleting (preconditioning) chemotherapeutic. For example, methods of conditioning a patient in need of a T cell therapy comprising administering to the patient specified beneficial doses of cyclophosphamide (between 200 mg/m²/day and 2000 mg/m²/day, about 100 mg/m²/day and about 2000 mg/m²/day; e.g., about 100 mg/m²/day, about 200 mg/m²/day, about 300 mg/m²/day, about 400 mg/m²/day, about 500 mg/m²/day, about 600 mg/m²/day, about 700 mg/m²/day, about 800 mg/m²/day, about 900 mg/m²/day, about 1000 mg/m²/day, about 1500 mg/m²/day or about 2000 mg/m²/day) and specified doses of fludarabine (between 20 mg/m²/day and 900 mg/m²/day, between about 10 mg/m²/day and about 900 mg/m²/day; e.g., about 10 mg/m²/day, about 20 mg/m²/day, about 30 mg/m²/day, about 40 mg/m²/day, about 40 mg/m²/day, about 50 mg/m²/day, about 60 mg/m²/day, about 70 mg/m²/day, about 80 mg/m²/day, about 90 mg/m²/day, about 100 mg/m²/day, about 500 mg/m²/day or about 900 mg/m²/day). A preferred dose regimen involves treating a patient comprising administering daily to the patient about 300 mg/m²/day of cyclophosphamide and about 30 mg/m²/day of fludarabine for three days prior to administration of a therapeutically effective amount of engineered T cells to the patient.

In some embodiments, lymphodepletion further comprises administration of a CD52 antibody. In some embodiments, the CD52 antibody is alemtuzumab. In some embodiments, the CD52 antibody is administered at a dose of about 13-30 mg/day IV.

In other embodiments, the antigen binding domain, transduced (or otherwise engineered) cells and the chemotherapeutic agent are administered each in an amount effective to treat the disease or condition in the subject.

In some embodiments, compositions comprising CAR-expressing immune effector cells disclosed herein can be administered in conjunction with any number of chemotherapeutic agents, which can be administered in any order. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PROD; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RF 52000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone.

In some embodiments, the chemotherapeutic agent is administered at the same time or within one week after the administration of the engineered cell, polypeptide, or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell, polypeptide, or nucleic acid. In other embodiments, the chemotherapeutic agent is administered at least 1 month before administering the cell, polypeptide, or nucleic acid. In some embodiments, the methods further comprise administering two or more chemotherapeutic agents.

A variety of additional therapeutic agents can be used in conjunction with the compositions described herein. For example, potentially useful additional therapeutic agents include PD-1 inhibitors such as nivolumab (Opdivo®), pembrolizumab (Keytruda®), pembrolizumab, pidilizumab, and atezolizumab (Tcentriq®).

Additional therapeutic agents suitable for use in combination with the disclosure include, but are not limited to, ibrutinib (Imbruvica®), ofatumumab (Arzerra®, rituximab (Rituxan®), bevacizumab (Avastin®), trastuzumab (Herceptin®), trastuzumab emtansine (KADCYLA®, imatinib (Gleevec®), cetuximab (Erbitux®, panitumumab) (Vectibix®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In some embodiments, the composition comprising CAR-containing immune cells can be administered with a therapeutic regimen to prevent cytokine release syndrome (CRS) or neurotoxicity. The therapeutic regimen to prevent cytokine release syndrome (CRS) or neurotoxicity can include lenzilumab, tocilizumab, atrial natriuretic peptide (ANP), anakinra, iNOS inhibitors (e.g., L-NIL or 1400W). In additional embodiments, the composition comprising CAR-containing immune cells can be administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

In certain embodiments, the compositions described herein are administered in conjunction with a cytokine. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1 alpha, IL-2, IL-3, IL-4, IL-5, IL-6, IL-7, IL-8, IL-9, IL-10, IL-11, IL-12; IL-15, IL-21 a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

### Cell Culture Model of HHV-6 Latent Infection and Reactivation

Cell lines with at least partial HHV-6 genomes integrated into the host cell genome have been reported for studying HHV-6 integration. But no evidence of viral reactivation and expression of a spectrum of HHV-6 genes after reactivation has been shown in these cell lines. Thus, the utility of these cell lines for investigating HHV-6 reactivation has not been demonstrated.

Thus, in another aspect, provided herein is an in vitro cell culture model for HHV-6 latent infection and reactivation. In one related aspect, the instant disclosure provides methods of establishing an in vitro cell culture model for HHV-6 latent infection and reactivation. In some embodiments, the cells are human lymphoid cells. In some embodiments, the cell culture model can be derived from or generated by using a human lymphoid cell line, including without limitation, CEM, Molt-3, Jhan or HSB2 cells. In some embodiments, the method comprises steps of infecting human lymphoid cells with HHV-6; and serially passaging the infected cells, thereby generating the cell culture model or cell line of HHV-6 latent infection and reactivation. In some embodiments, the human lymphoid cells are infected by HHV-6 at a multiplicity of infection (m.o.i.) of no more than about 10, no more than about 9, no more than about 8, no more than about 7, no more than about 6, no more than about 5, no more than about 4, no more than about 3, no more than about 2, no more than about 1, or no more than about 0.5 m.o.i. The infected cells are maintained in the cell culture for a period of time before being subjected to serial passaging. In some embodiments, the infected cells are maintained in the cell culture and monitored until the virus reaches logarithmic growth in the cells. In some embodiments, the infected cells are maintained in the cell culture for about 12 to about 19 days before subjected to serial passaging. In some embodiments, the infected cells are maintained in the cell culture until the virus reaches logarithmic growth in the culture before subjected to serial passaging. In some embodiment, increments of the viral DNA in log scale of 10 as detected by qPCR is indicative of logarithmic growth.

In some embodiments, the infected cells are serially passaged in cell culture for a period of time. In some embodiments, the cells are serially passaged in cell culture to dilute the cell density and/or avoid confluence of the cell culture. In some embodiments, the cells are serially passaged in cell culture to maintain a cell density in the cell culture of no more than about 1×10⁶ cells/cm², no more than about 0.9×10⁶ cells/cm², no more than about 0.8×10⁶ cells/cm², no more than about 0.7×10⁶ cells/cm², no more than about 0.6×10⁶ cells/cm², no more than about 0.5×10⁶ cells/cm², no more than about 0.4×10⁶ cells/cm², no more than about 0.3×10⁶ cells/cm², no more than about 0.2×10⁶ cells/cm², or no more than about 0.1×10⁶ cells/cm², or about 0.2×10⁶ cells/cm² to about 0.9×10⁶ cells/cm², about 0.2×10⁶ cells/cm² to about 0.8×10⁶ cells/cm², about 0.2×10⁶ cells/cm² to about 0.7×10⁶ cells/cm², about 0.2×10⁶ cells/cm² to about 0.6×10⁶ cells/cm², or about 0.2×10⁶ cells/cm² to about 0.5×10⁶ cells/cm². In some embodiments, the cells are serially passaged for about 20 days, about 25 days, about 30 days, about 35 days, about 40 days, about 45 days, about 50 days, about 55 days, about 60 days, about 65 days, about 70 days, about 75 days, or about 80 days. In some embodiments, the method further comprises the step of detecting HHV-6 viral DNA or viral RNA, wherein a constant level of HHV-6 DNA or an absence of detectable HHV-6 RNA indicates HHV-6 latent infection.

In some embodiments, the HHV-6 latent infection cell culture model comprises cells that harbor about 10² to about 10³, about 10² to about 10⁴, or about 10³ to about 10⁴ copies of HHV-6 genome equivalents per 500 ng extracted genomic DNA, or no more than about 10², no more than about 10³, or no more than about 10⁴ copies of HHV-6 genome equivalents per 500 ng extracted genomic DNA, without induction of reactivation. In some embodiments, the viral genome equivalents can be calculated by extrapolation by comparing the amount of the DNA of a viral gene detected in a sample against a standard curve established based on predetermined amounts of the DNA of the viral gene in a plasmid. In some embodiments, the viral gene is present in one copy of the viral genome, such as U31 or U65-66.

In some embodiments, the HHV-6 latent infection cell culture model comprises cells that harbor detectable HHV-6 DNA, as measured by methods known in the art and/or described herein, and do not show detectable HHV-6 RNA transcript, as measured by methods known in the art and/or described herein. In some embodiments, the HHV-6 genome copy number is determined by detecting U31 or U65-66 viral DNA by, e.g., qPCR. In some embodiments, the HHV-6 latent infection and reactivation cell culture model comprises cells that do not exhibit detectable HHV-6 transcripts as determined by, e.g., RT-qPCR.

In some embodiments, the cell culture model is inducible of HHV-6 immediate early genes, early genes, and/or late genes expression upon the induction of reactivation. In some embodiments, the cell culture model is inducible of one or more HHV-6 genes U95, U86, U39, U54, U14, U79-2 U24 or U46 expression. In certain embodiments, the cell culture model can be induced to reactivate latent HHV-6 infection by sodium butyrate and PMA.

The in vitro cell culture model allows ways to induce reactivation of latent HHV-6 infection in a controlled manner and can be useful for multiple applications. For example, using this cell culture model, one can control the timing and extent of HHV-6 reactivation and can be useful for understanding HHV-6 biology and for conducting anti-viral drug screening. Thus, in a related aspect, disclosed herein are methods of screening for agents, inhibitors, or compounds that can affect HHV-6 replication, latency and/or reactivation, comprising the steps of contacting the agents, inhibitors, or compounds with the cell culture model and analyzing the effects thereof on viral latent infection, reactivation, replication and/or lytic or active infection. In a further aspect, disclosed here are methods of determining the optimal amount of an agent, inhibitor, or compound for inhibiting HHV-6 reactivation, comprising the steps of contacting the cells with different amounts of the agent, inhibitor or compound and analyzing the effects thereof on viral latent infection, reactivation, replication and/or lytic or active infection. In a related aspect, disclosed here are methods of determining the optimal timing of applying an agent, inhibitor, or compound for inhibiting HHV-6 reactivation, comprising the steps of contacting the cells with the agent, inhibitor or compound and analyzing the effects thereof on viral latent infection, reactivation, replication and/or lytic or active infection. In some embodiments, the agent comprises a CAR T cell or CAR T cells specific for HHV-6. In some embodiments, the effects or degrees of effects can indicate the potency of the CAR T cells specific for HHV-6. In some embodiments, the effects are changes to the level of viral DNA replication, and/or changes to the level of viral RNA transcription, and/or changes to the level of protein expression, by methods described herein or known in the art.

All references cited herein, including patents, patent applications, papers, text books, and the like, and the references cited therein, to the extent that they are not already, are hereby incorporated by reference in their entirety.

The following examples are offered for illustrative purposes only. Various modifications of the invention in addition to those shown and described herein will become apparent to those skilled in the art from the foregoing description.

### EXAMPLES

### Example 1 IFNα Treatment Effectively Reduced HHV-6 Viral Load in CAR T Cells

Drug product (DP) for experiment was selected based on the results of screening for HHV-6, among other adventitous agents, at the end of cell engineering process. Batch #1 was one of a few rare occurrences of DP with “positive” levels of HHV-6 and was chosen for the following studies (designated as the “HHV-6 high” group), while Batch #2 was chosen as the “low”-level counterpart. DP vials were retrieved from LN2, thawed and each split into 4 day 0 (D0) flasks. We first tested the effects of a type I IFN, such as IFNα, on the replication and infectivity of HHV-6. Flasks received either complete media (X-vivo +5% human serum (HS)+supplements) or complete media with added IFNα (human IFN-α2a at 100 ng/mL, 1 ng/mL and 0.01 ng/mL, Miltenyi). Cells were cultured for 7 additional days (D1-D7), with daily collections of cells from all conditions for freezing. Media was replaced every three days, with or without added IFNα for corresponding flasks.

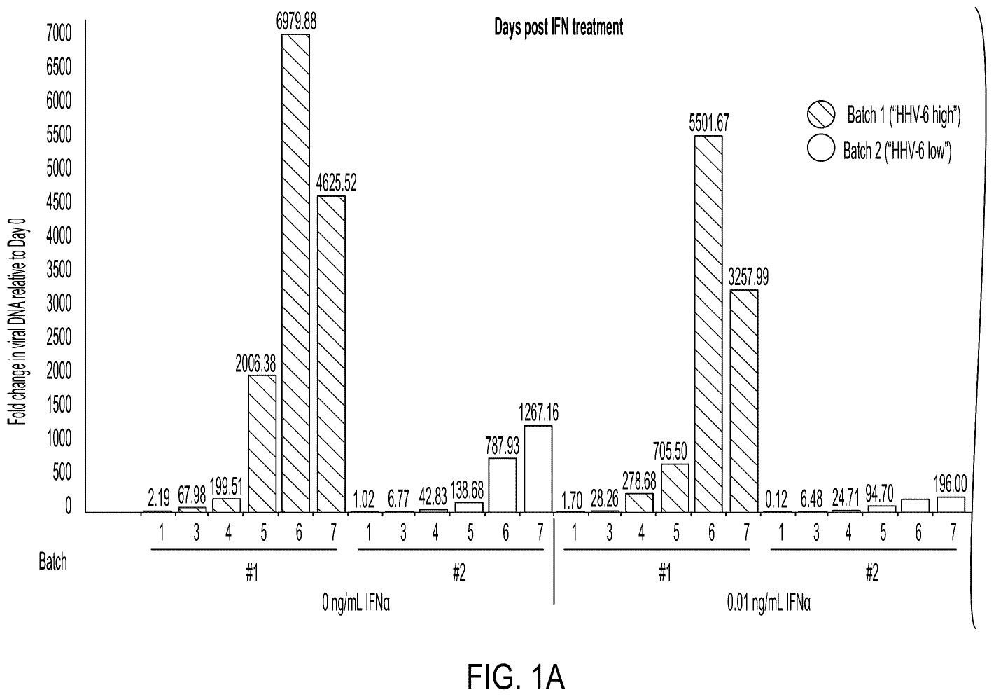

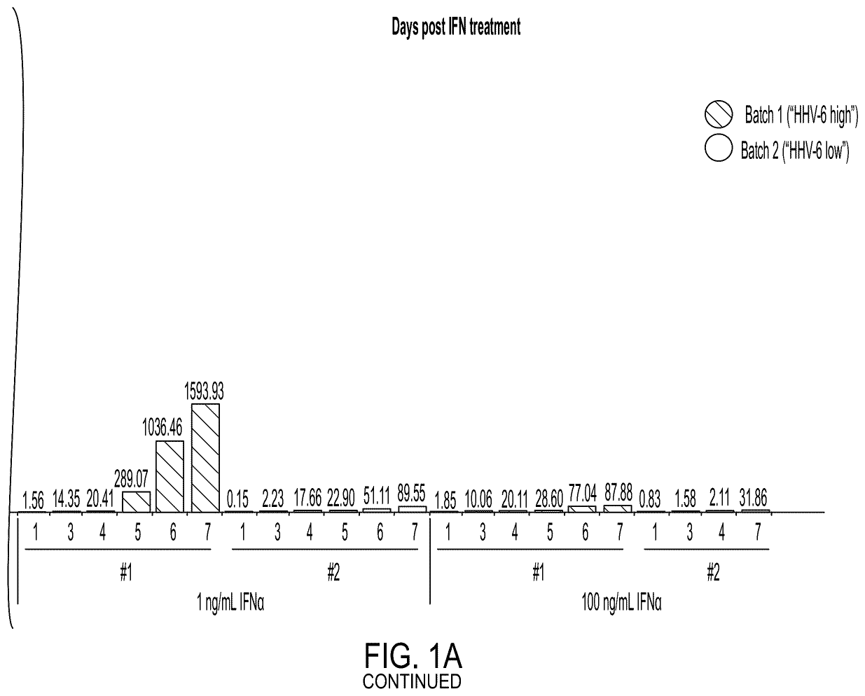

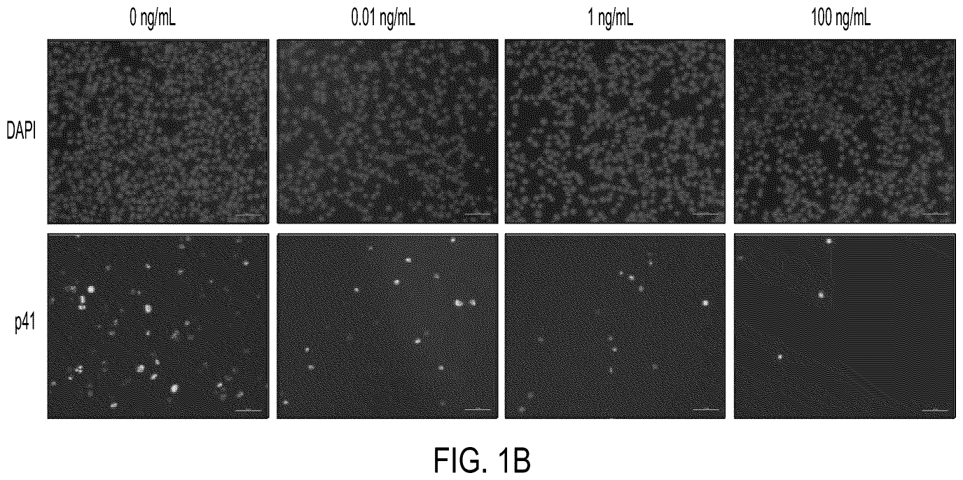

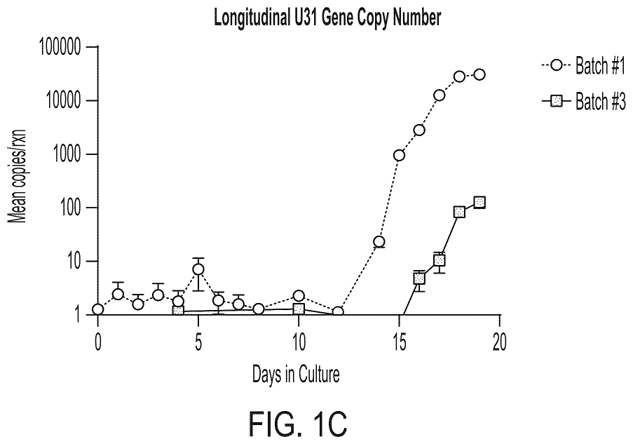

Post-cell culture, DNA was extracted from the collected samples, and analyzed by qPCR for HHV-6 marker U31 as described in Material & Methods. The data in FIG. 1A shows the fold-change of HHV-6 viral DNA from D1-D7 of all eight conditions (two batches of DPs, each with 3 IFNα concentrations and a non-treated control) relative to its D0 counterpart. Batch 1 in the control group without IFNα treatment shows a significant increase in HHV-6 viral DNA from D1 to D6, while the “HHV-blow” DP (batch 2) shows a much lesser increase during the same time period. For both lots of DP, low doses of IFNα led to a reduction in the viral load compared to the control; the reduction was even more evident at the higher doses of IFNα.

To test whether HHV-6 protein expression is affected by IFNα treatment, we performed immunofluorescence using an antibody specific for HHV-6 p41, an HHV-6 early gene product, on cells collected from D4 of the Batch 1 (“HHV-6 high”) DP culture. As shown in FIG. 1B, with increasing concentration of IFNα we observed fewer cells positive for HHV-6 p41 in the culture. For the batch 2 (“HHV-6 low”) culture, only few HHV-6 p41 positive cells were observed on D4 even with no IFNα treatment, while IFNα treatment led to no detectable HHV-6 p41 positive cells regardless of concentration used (data not shown). Thus, IFNα effectively reduced not only HHV-6 viral loads but also viral protein expression in engineered CAR T cells.

The samples of two rare occurences of HHV-6 positive DPs (“HHV-6 high” or “HHV-blow”) were subjected to longitudinal analysis retroactively to detect the viral DNA levels during cell culture in the manufacturing process. Both DP samples exhibited extremely low or non-detectable levels of U31 DNA by qPCR at the early time points of cell culture (FIG. 1C). An increase in U31 DNA levels at later time points was observed in both the “HHV-6 high” sample (Batch #1) and a “HHV-6 low” sample (Batch #3).

Based on these results, we next developed experimental models to investigate how IFN can be used to suppress reactivation of latent virus, such as HHV-6, in cell culture of engineered immune cells, such as CAR T cells.

### Example 2 Establishment of an HHV-6 Latent Infection and Reactivation Cell Culture Model

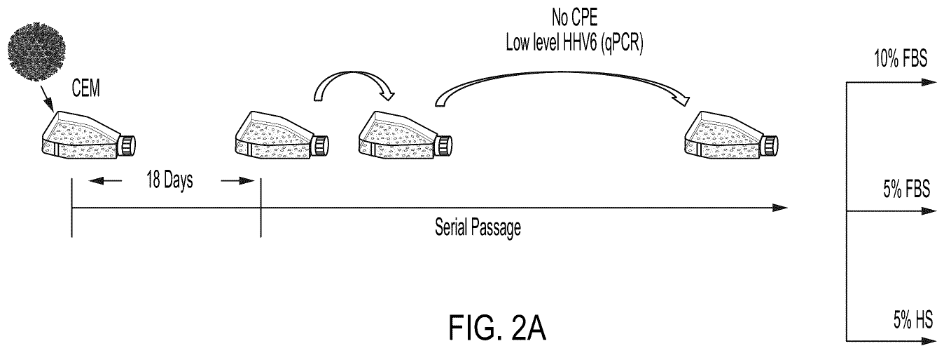

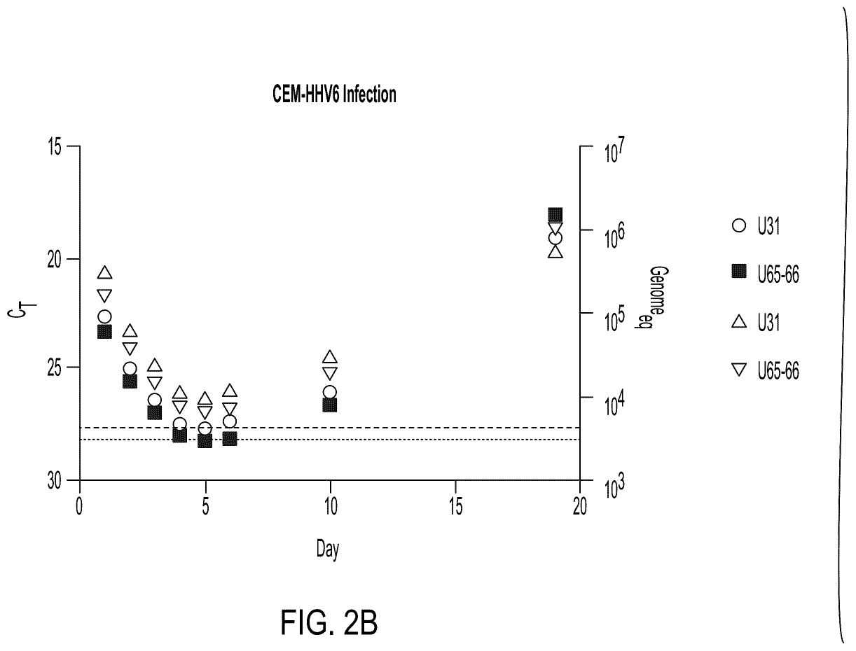

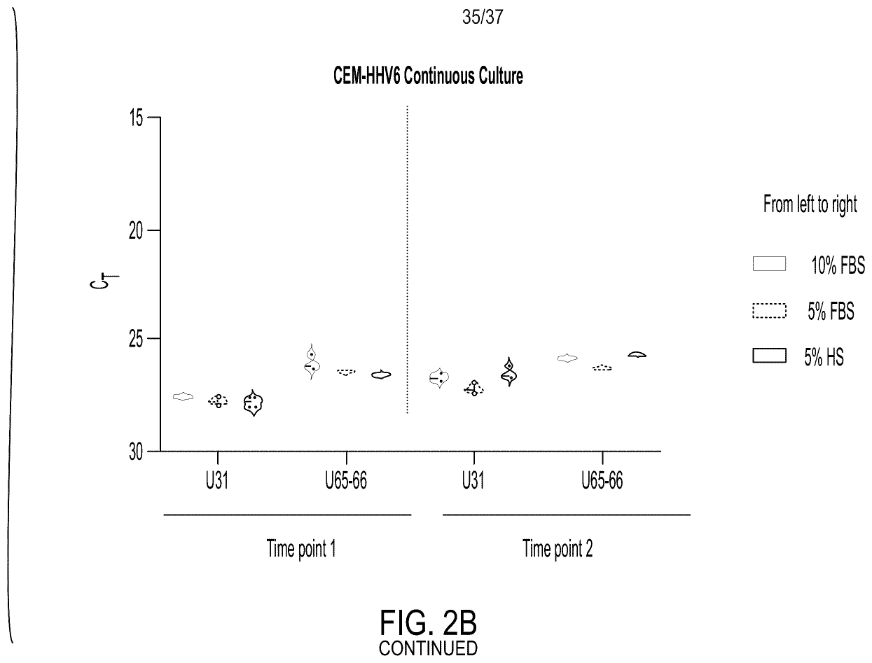

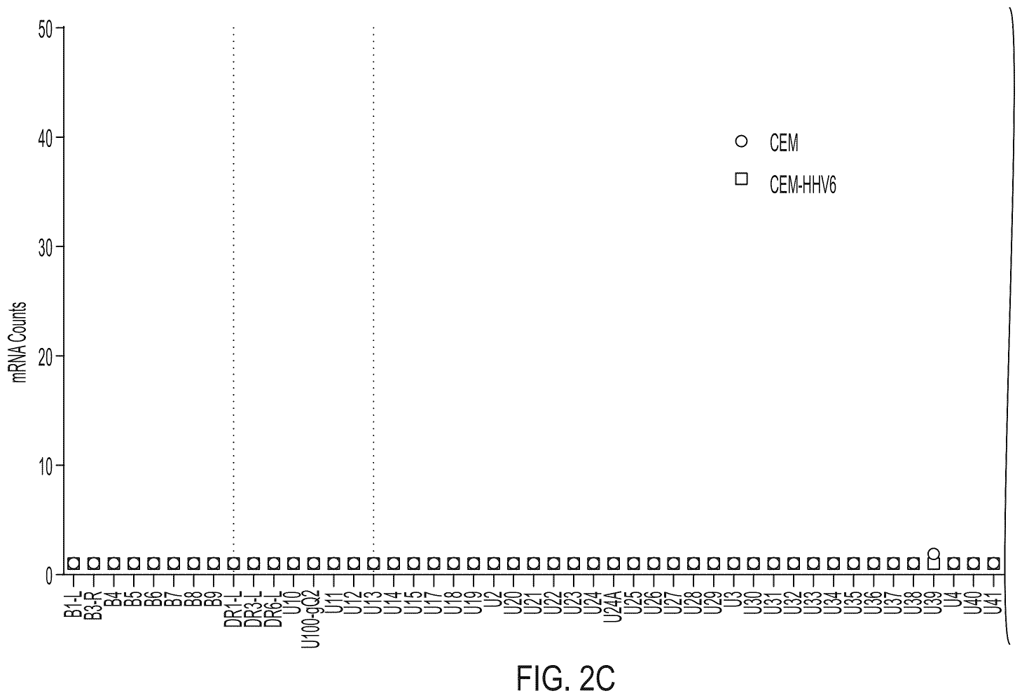

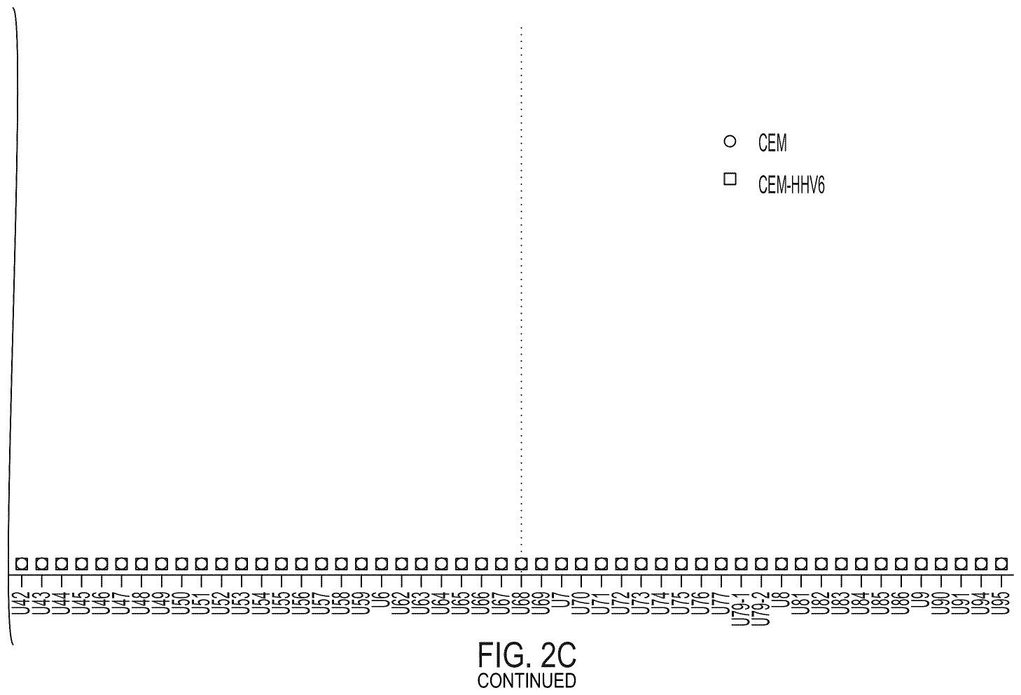

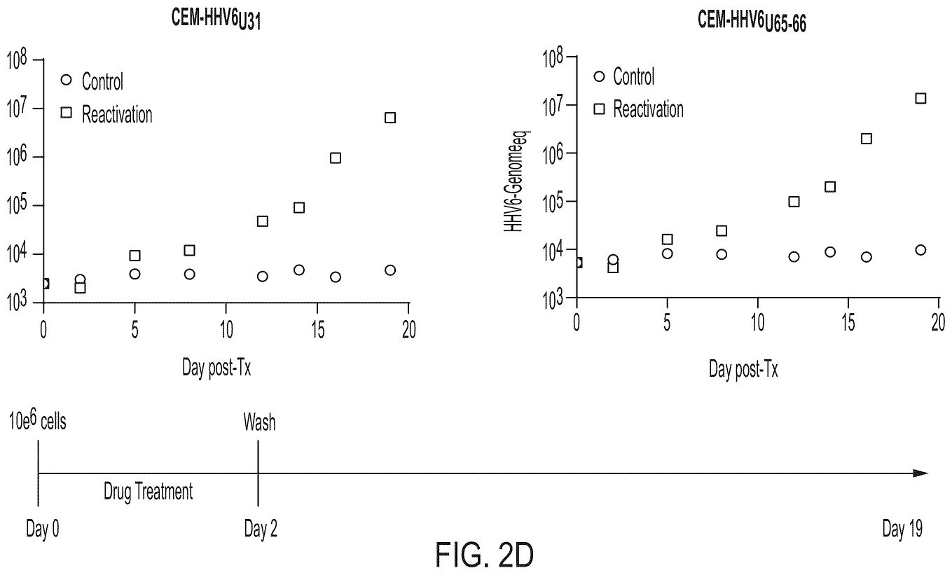

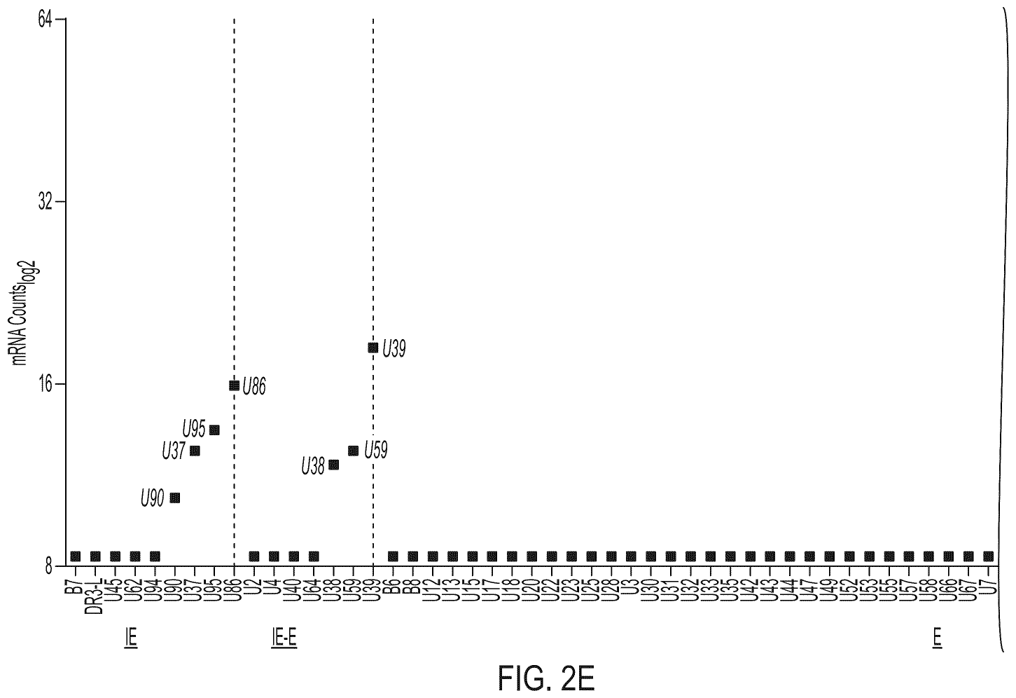

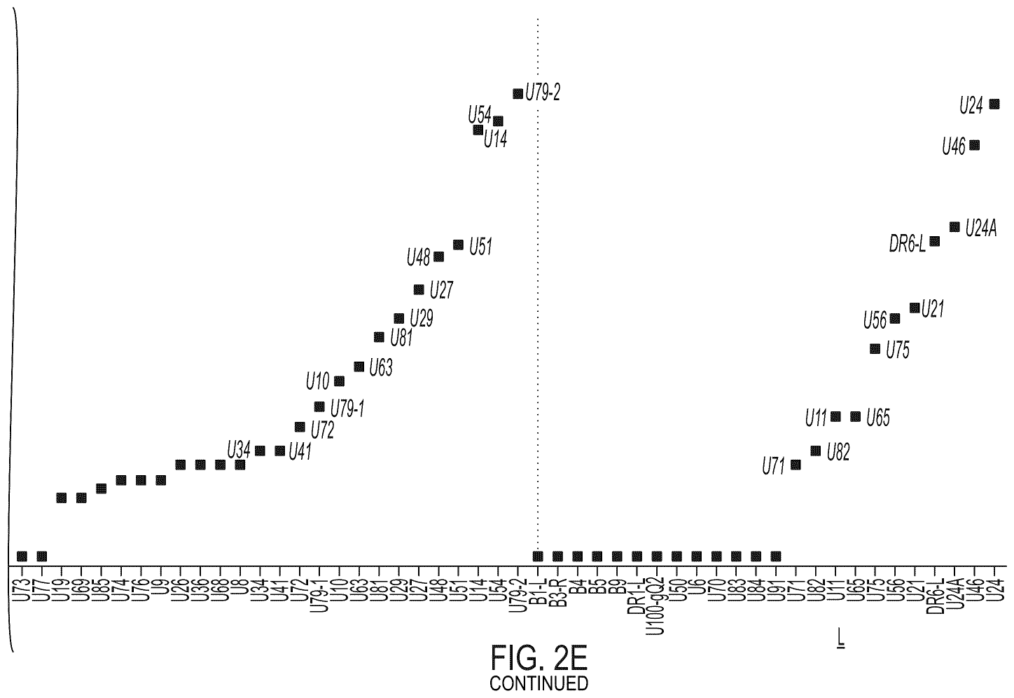

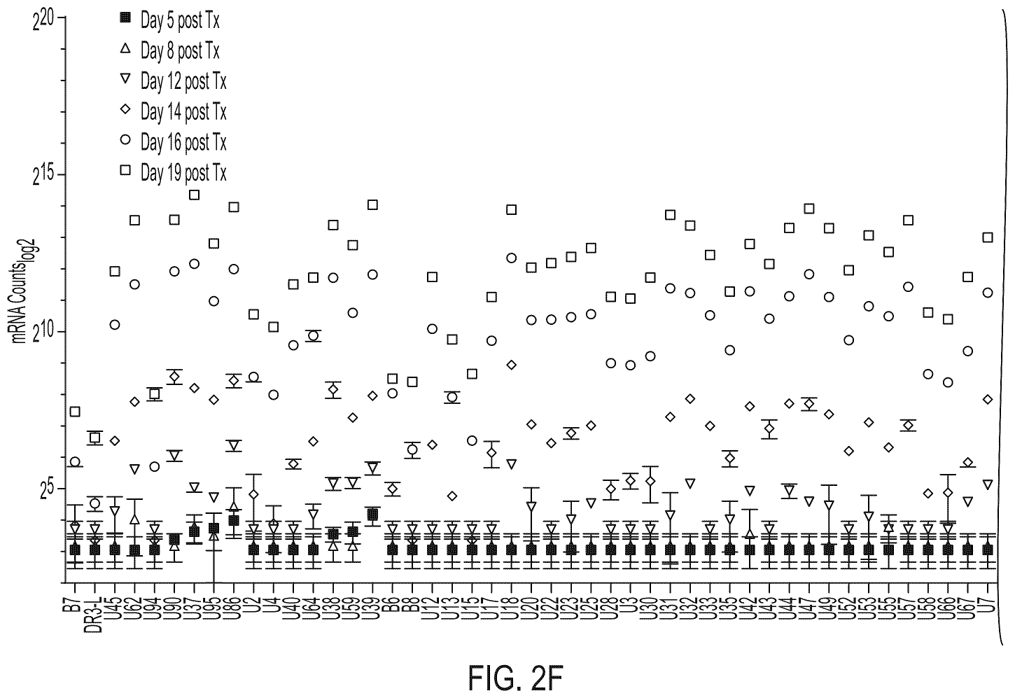

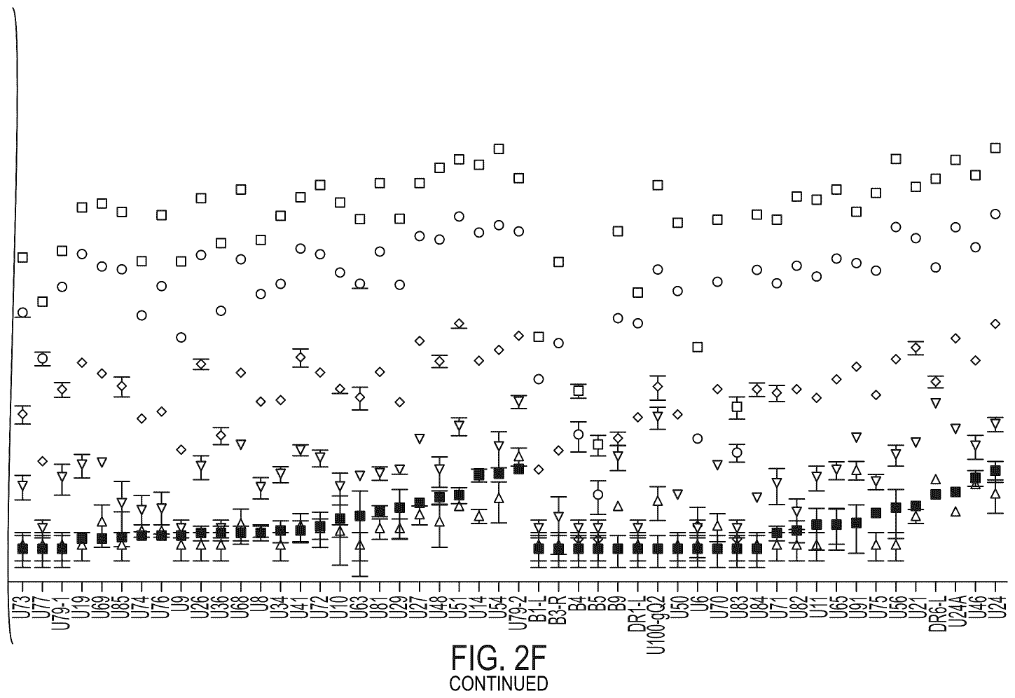

To our knowledge, currently there is no suitable cell culture model for HHV-6 latency and reactivation. Cell lines with at least partial HHV-6 genome integrated into the chromosome have been reported. But the utility of the cell lines for studying latent viral infection, viral reactivation and control thereof has not been demonstrated. See e.g., Gulve et al. 2017, Chromosomal integration of HHV-6A during non-productive viral infection. Nature, DOI:10.1038/s41598-017-00658-y. We established an in vitro cell culture model to facilitate the investigation of ways to control viral reactivation. The human lymphoid CEM cells were utilized to establish the latent infection model with human herpesvirus 6B (strain Z-29). CEM cells (2×10⁷ cells) were infected with 100 uL of HHV-6B supernatant in RPMI media containing 5% human serum (HS), 5% or 10% fetal bovine serum (FBS) and incubated at 37° C. for 4 hours. Infected CEM cells were then washed and resuspend in the appropriate media over the course of 19 days. Cells were collected on days 1-6, 10 and 19 for qPCR analysis. To establish latent infection in the cells, on day 19, after observing viral logrithmic growth, infected CEM cells were split 1 to 3 by volume and kept in culture for ˜2 months, splitting 1 to 3 every third day and maintaining low cell density in culture dishes until HHV-6B detection remained constant by qPCR. Genomic DNA was extracted from the collected samples and analyzed for viral infection using the primer probes for detecting the U31 and U65-66 viral genes. Viral adsorption was observed up to day 5 post infection, and an increase of viral genomic DNA (vgDNA) was observed at days 10 and 19 indicating logrithmic growth of the virus (FIG. 2B, left panel). U31 (circle) and U65-66 (square) Ct values are reported on left Y axis and viral genome equivalents based on U31 (triangle) and U65-66 (inverted triangle) on the right Y-axis (FIG. 2B, left panel). The dashed lines represent the adopted baseline of latent infection as determined by U31 DNA (the lower dashed line) or U65-66 DNA (the higher dashed line). qPCR analysis of the continuous culture of the HHV-6B infected CEMs (CEM-HHV6) show no increase in Ct values following a week in culture indicating that we had established a latent infection (FIG. 2B, right panel). HHV-6B latency was confirmed in the CEM-HHV6 model by analyzing the expression of HHV-6B viral transcripts using the nCounter® system (nanoString) (FIG. 2C). The expression of 97 viral gene transcripts were analyzed from total RNA extracted from CEM (negative control) and CEM-HHV6 cells. Despite detection of constant viral DNA levels in the cells (FIG. 2B, right panel), no detectable viral transcripts were observed in the continuous coulture (FIG. 2C), which is indicative of viral latency.

We next tested whether viral reactivation can be induced in the latency cell culture model. CEM-HHV6 cells (1×10⁷) were reactivated using 100 ng/mL of PMA (phorbol-12-myristate-13-acetate, Cat #500582 Millipore-Sigma) and 1 mM NaButyrate (Cat #19-137, Millipore-Sigma) final concentration for 2 days at 37° C. On day 2 cells were washed and resuspended in RPMI+5% FBS and cultured for 18 days. Media was replenished as needed. CEM-HHV6 cells were collected on days 5, 8, 12, 14, 16, and 19 post-reactivation. HHV-6B viral reactivation was assessed by qPCR analysis of the vgDNA of HHV-6B targeting the U31 and U65-66 genes. Untreated CEM-HHV6 cells were used as a negative control. As shown in FIG. 2D, increased level of vgDNA were evident beginning day 5 post-reactivation and continued to increase exponentially up to the last collected time-point; compared to untreated CEM-HHV6 cells. HHV-6B reactivation was further assessed by extracting total RNA from the reactivated CEM-HHV6 samples and analyzing viral gene expression with the nCounter® system. Several viral RNA transcripts, including immediate early gene U19 and early gene U69, were detectable as early as day 5 post-treatment (FIG. 2E) and continued to increase over-time (FIG. 2F). Viral cytopathic effect (CPE) was also observed in the reactivated culture (data not shown). FIG. 2E shows expression profile of several immediate early (IE), immediate early early (IE-E), early I and late (L) genes, resembling that of the beginning of an active infection after reactivation, and FIG. 2F show progression to active infection. These results are consistent with the increase of HHV-6B vgDNA, and are indicative of the reactivation of the latent HHV-6B virus in the CEM-HHV6 cell model. The CEM-HHV6 cells remained stable after at least four or five passages and after cell banking. Thawed cells from cell bank were kept in culture for at least 30 days and were able to retain the phenotype of HHV-6 latent infection as determined by qPCR (data not shown).

### Example 3 IFNα Effectively Repressed HHV-6 Replication in the CEM-HHV6 Cell Reactivation Model

The CEM-HHV6 reactivation model described in FIGS. 2A-F was utilized for testing the effects of IFNα in the cell culture model, similar to the testing carried out in DP (FIG. 1A). CEM-HHV6 cells were initially cultured in bulk before the start of the experiment. One day before the start of the experiment (D-1), an aliquot of the CEM-HHV6 cells was taken out and split before treatment with one of three IFNα concentrations (100 ng/mL, 1 ng/mL and 0.01 ng/mL), which constituted the “pre-treatment” of IFNα. Cells were collected from the beginning of the experiment (D0) and continuing every few days throughout. At D3, the reactivation treatment (RA) described in FIG. 2D was applied to aliquots of cells that had undergone pre-treatment, as well as those that had not. From each of these samples, cells were retained for growth that did not undergo reactivation (NRA). On D4, cells from all groups (RA/NRA and pre-treatment/no pre-treatment) were subdivided further and some cells were treated with IFNα after reactivation (“post-treatment”).

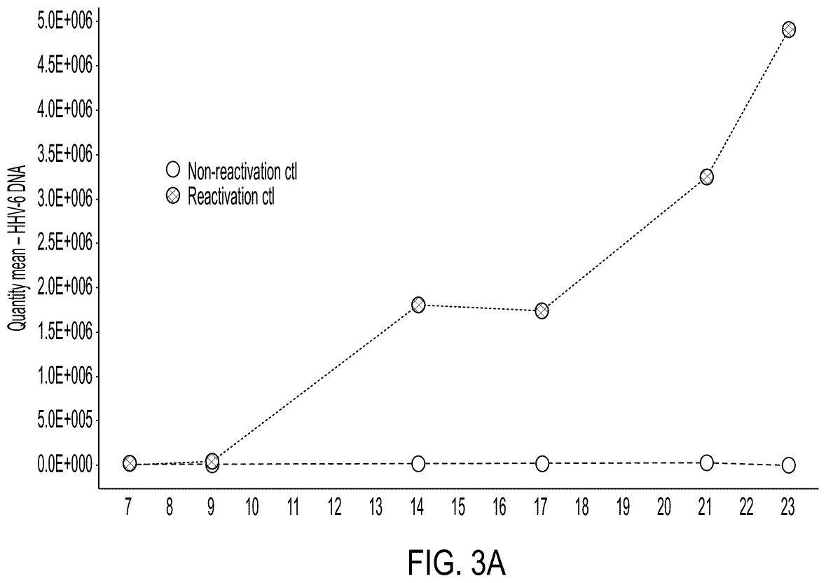

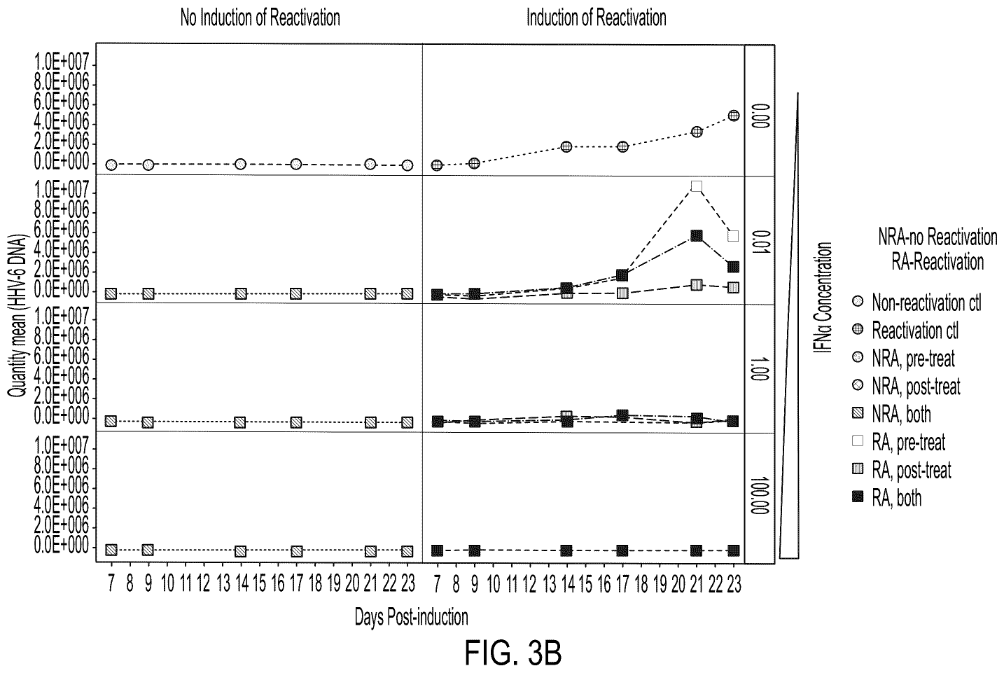

The results of this extensive series of treatments generated a large quantity of data supporting the conclusion from the experiment done with CAR T DP described above. All cells were subjected to DNA extraction, followed by HHV-6 qPCR as described in Material & Methods. In this cell culture model, HHV-6 reactivation was demonstrated by the increase of viral DNA post treatment with the reactivation agents sodium butyrate and PMA (FIG. 3A). The data for the IFNα-treated samples (FIG. 3B) showed that, as the IFNα dosage increased, the level of viral growth was reduced, and at 100 ng/mL, there was virtually no distinction between the NRA and the RA samples, showing that IFNα is counteracting the effects of the RA treatment and repressed viral growth even during an induced reactivation event.

### Example 4 IFNs Reduced HHV-6 Infectivity in a Small Scale CAR T Production Process

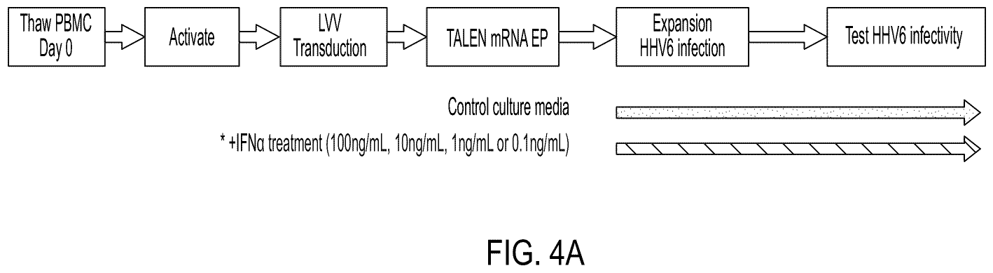

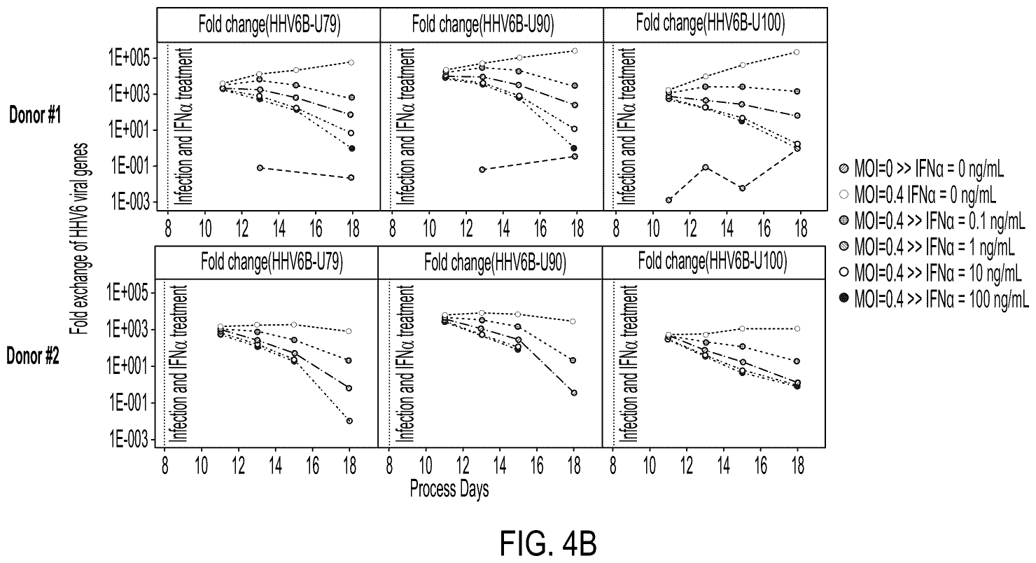

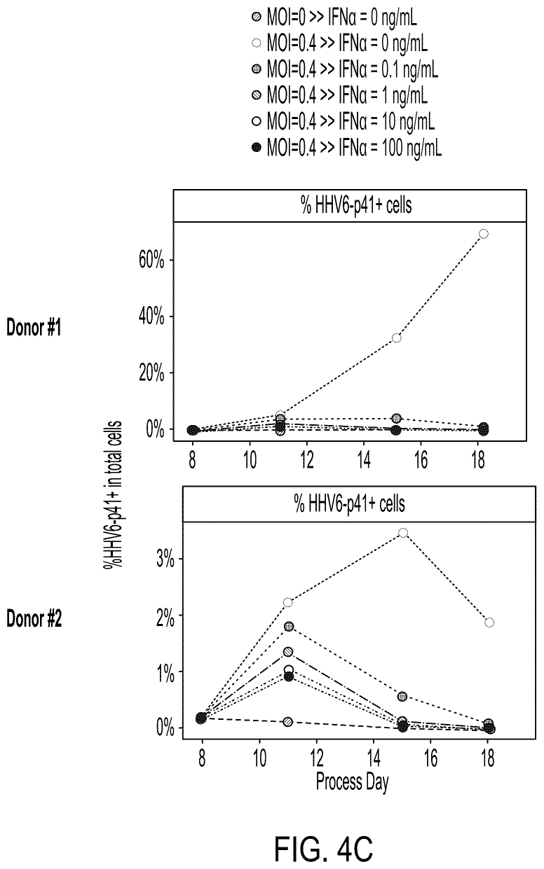

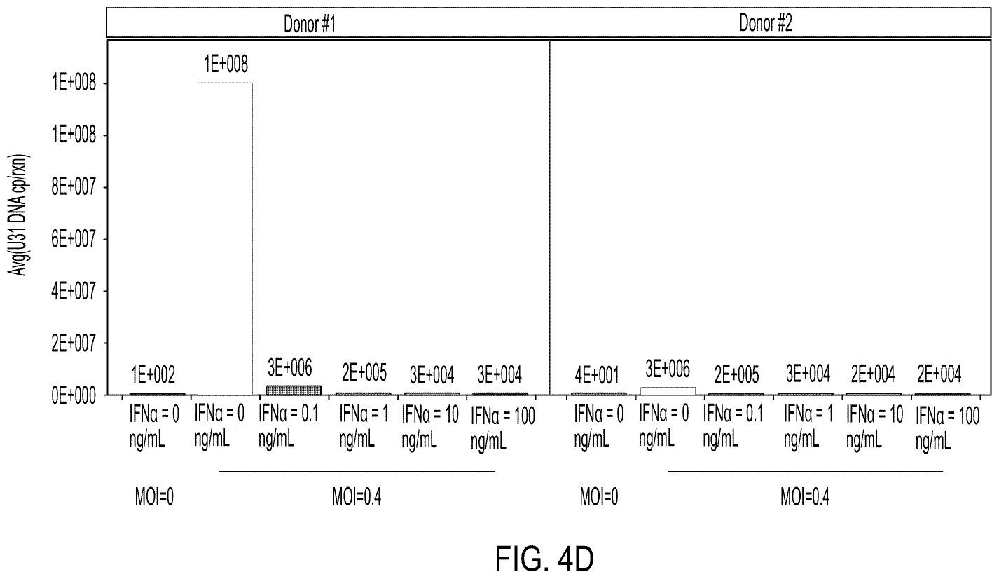

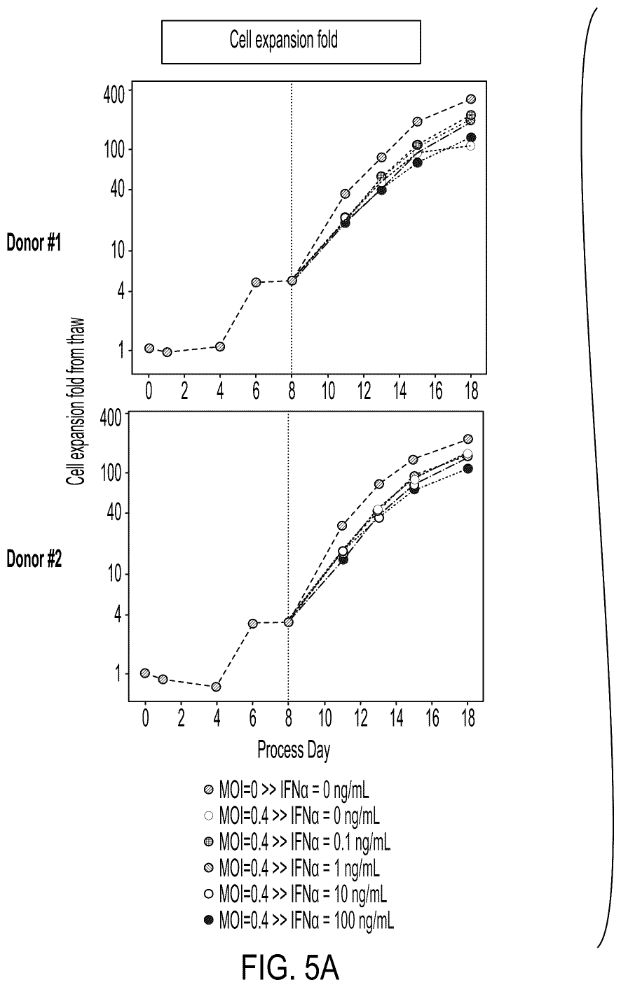

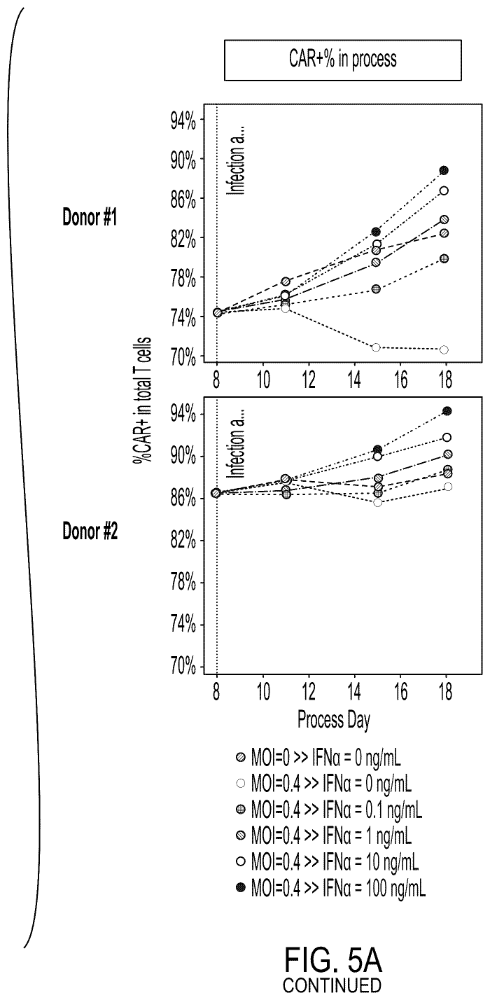

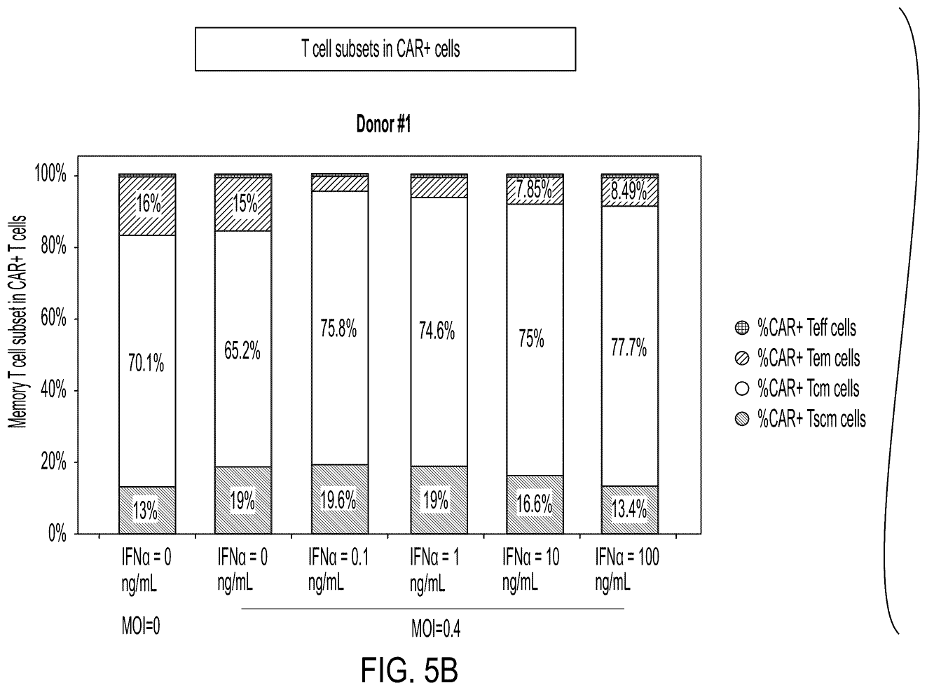

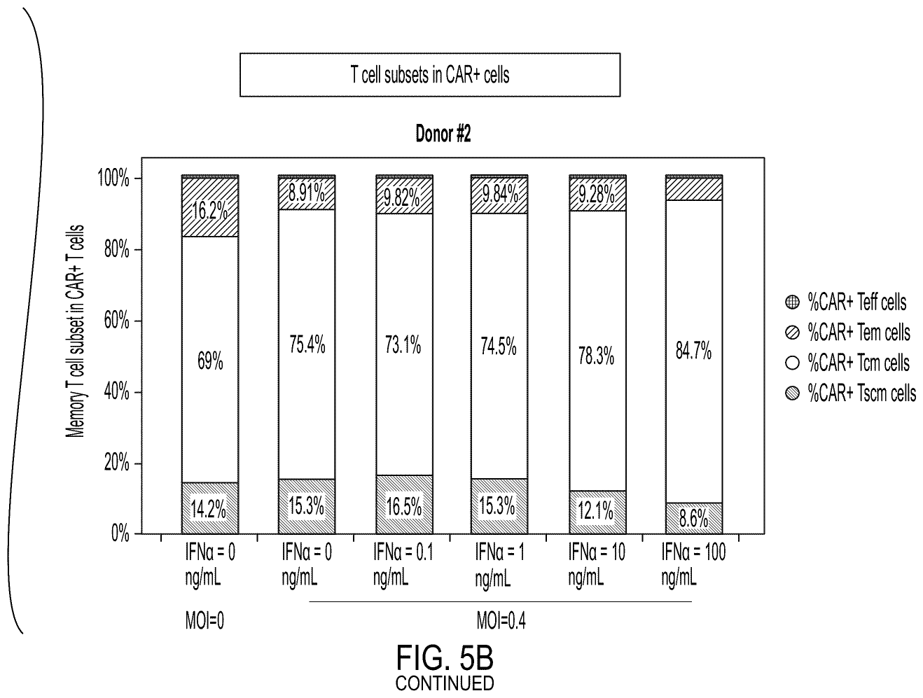

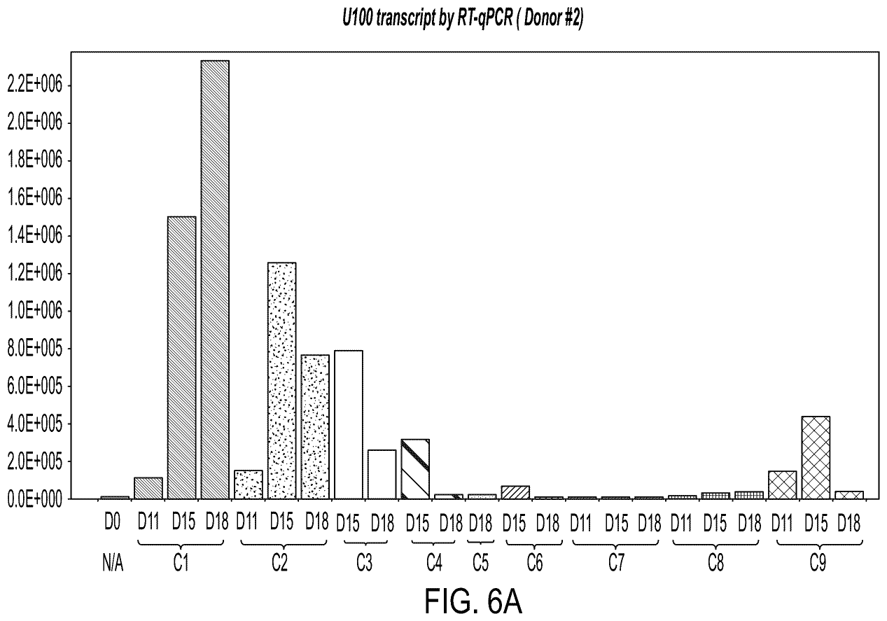

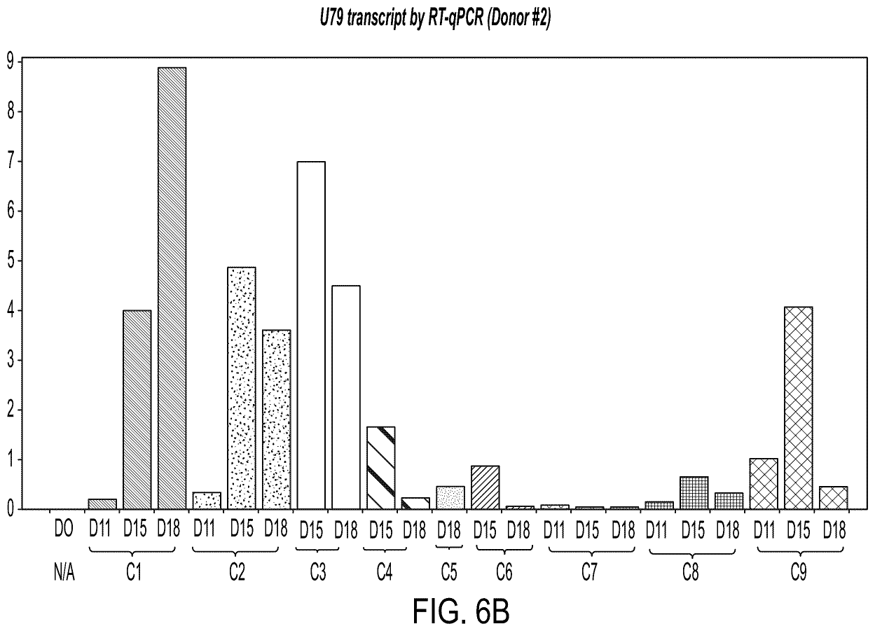

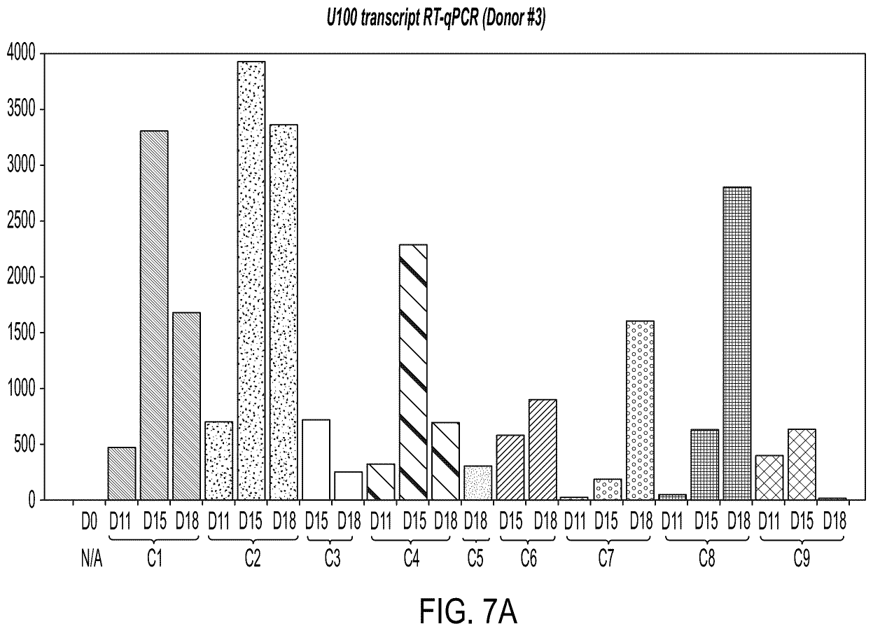

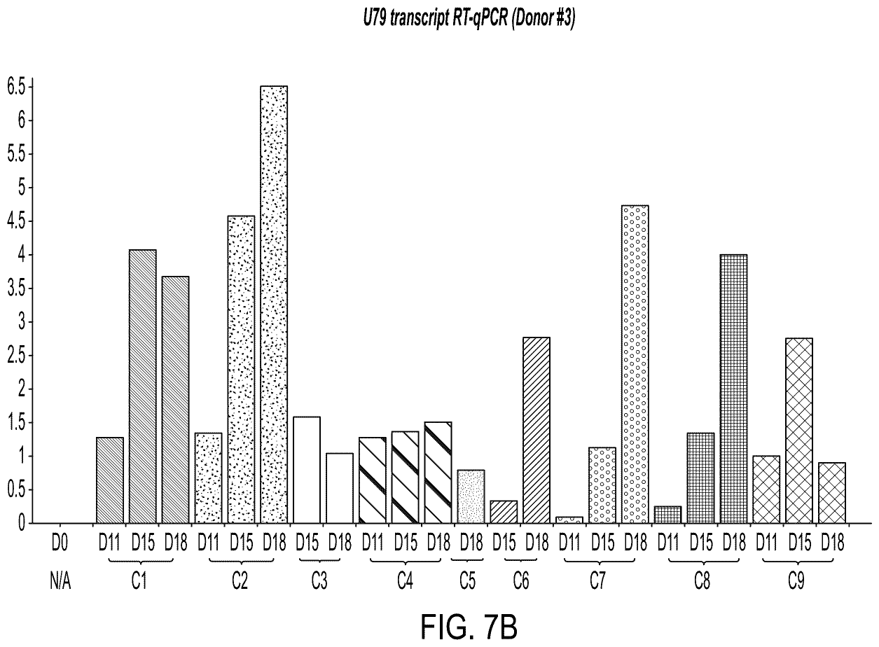

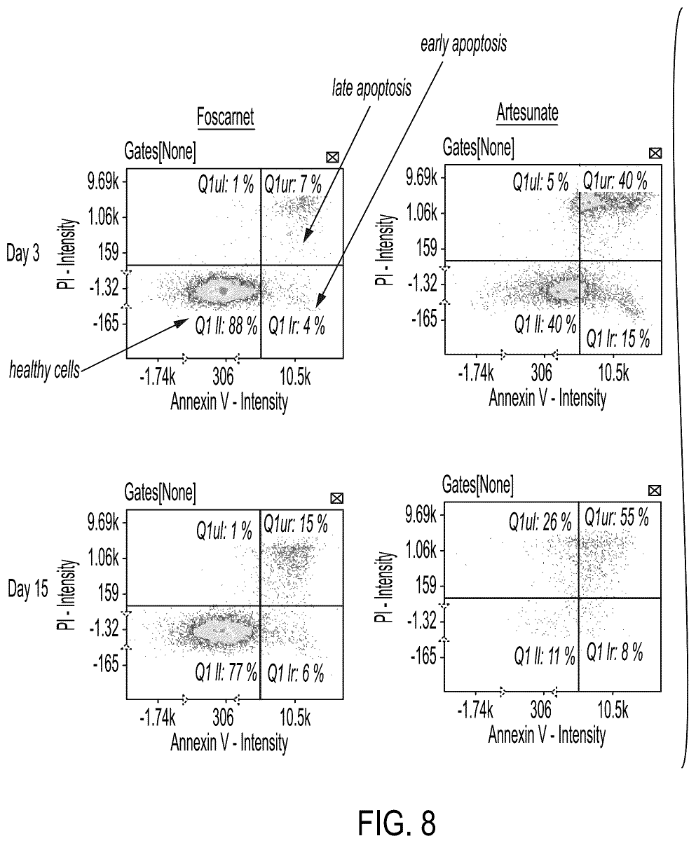

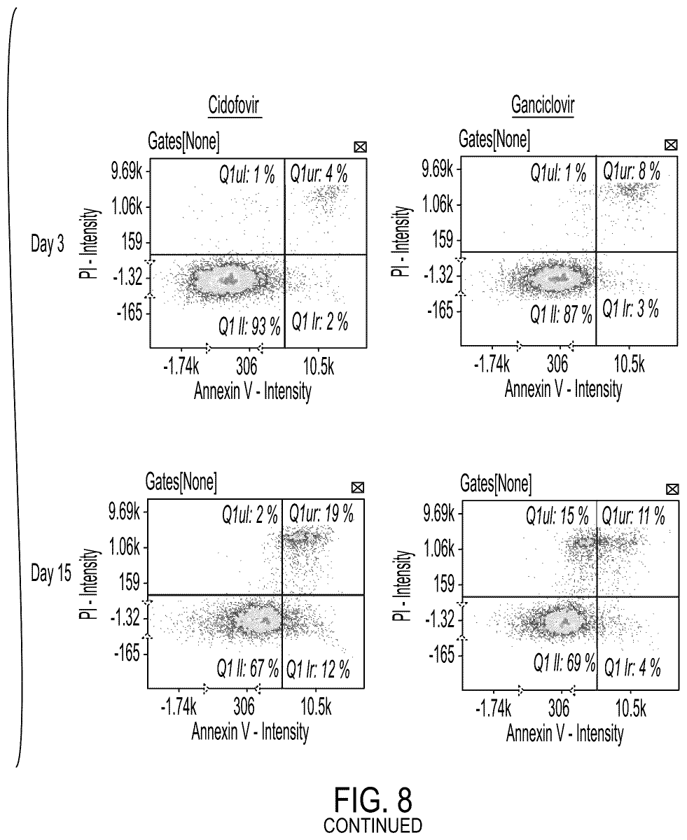

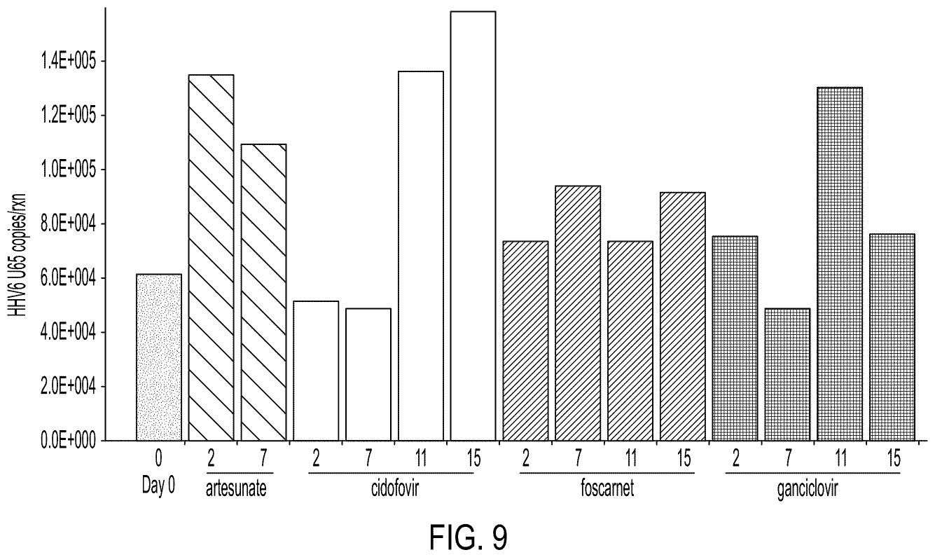

HHV-6 reactivation from PBMC is an extremely rare event in CAR T manufacturing based on existing data. To investigate the feasibility of using IFNα in controlling HHV-6 infectivity, we designed experiments to test the effects of IFNα in the manufacturing process by deliberately introducing active HHV-6 infection. The schematic representation of the experiment design is shown in FIG. 4A. In brief, the PBMCs from two different donors were thawed from frozen storage and cultured in complete media (5% HS+X-vivo+Supplements) for activation and LVV transduction. After electroporation of cells with TALEN mRNA, the T cells were infected with HHV-6B (strain Z-29) at MOI=0.4 for 3 hrs, then transferred to the G-Rex bioreactor for expansion for 10 days (Day 8 to Day 18). IFNα was added into the complete media at different concentrations (100 ng/mL, 10 ng/mL, 1 ng/mL and 0.1 ng/mL) on D8 after the 3 hrs of HHV-6 infection and kept in culture until the end of the expansion period. Cell samples were collected on D11, D13, D15 and D18, i.e., three, five, seven, and ten days post infection, to determine the HHV-6 virus infectivity by RT-qPCR assay (FIG. 4B), flow cytometry detection (FIG. 4C), and qPCR assay (FIG. 4D). Description of RT-qPCR assay, flow cytometry, and genomic DNA qPCR assay are shown in Materials & Methods. Based on the RNA expression level of HHV-6 viral genes U79, U90 and U100, both donors showed HHV-6 infection three days post infection (FIG. 4B). Without any IFNα treatment, HHV-6 RNA levels increased from D11 to D18, i.e., day 3 to day 10 post infection in donor #1, whereas the RNA levels decreased from D11 to D18 even with the lowest concentration of IFNα treatment. 10 ng/mL IFNα can keep HHV-6 viral RNA below detectable levels on D18. Furthermore, a similar dose-dependent reduction of HHV-6 viral replication was seen in both donor #1 and donor #2 after IFNα treatment, with donor #2 PBMCs showing lower HHV-6 infectivity than donor #1 to begin with. Flow cytometry data show that even the lowest concentration of IFNα tested (0.1 ng/mL) effectively reduced the HHV-6 viral antigen p41 expression on D18 in both donors (FIG. 4C). Additionally, the dose-dependent inhibition of IFNα treatment was observed on HHV-6 U31 DNA levels (FIG. 4D). By process D18, the lowest amount of IFNα tested (0.1 ng/mL) was able to decrease HHV-6 DNA levels by 16 to 38-fold. The results from the active infection experiment show that IFNα added upon HHV-6 infection effectively repressed HHV-6 infectivity, repressed viral spreading and prevented HHV-6 replication and amplication in donor PBMC-engineered CAR T cells in a small-scale manufacturing process, as demonstrated by viral gene expression and viral DNA replication. IFNα treatment will likely have the same effects on patient PBMC-derived CAR T cells as well.

### Example 5 Titration of IFNs Doses for Use in CAR T Manufacturing

At certain IFNα doses, IFNα treatment could suppress the proliferation of CAR-T cells (data not shown). To minimize the impact of IFNα on CAR-T cell proliferation and/or phenotype, we titrated the amount of IFNα to find the lowest effective treatment dose with minimal negative effects on CAR T expansion and phenotype in the HHV-6 infection model as described in Example 4. As shown in FIG. 5A, IFNα in the range of 0.1 ng/mL to 10 ng/mL, added to the culture from Day 8 to Day 18, i.e., day 0 to day 10 post infection, did not significantly affect the fold of CAR T cell expansion, however, percentage of CAR+ cells increased in the groups receiving higher doses of IFNα treatment (FIG. 5A). The CAR T cells with IFNα treatment also showed a younger phenotype (% CAR Tcm+% CAR Tscm cells), as compared to the group without HHV-6 infection and without IFNα treatment (FIG. 5B). Treatment with IFNα from 0.1 ng/mL to 1 ng/mL had less of an effect on the % CAR+, CAR T cell expansion, and CAR-T cell phenotype than the higher concentration groups.

### Example 6 The Timing of Adding IFNα to Cell Culture

The above data show that IFNα effectively inhibited HHV-6 infection of engineered CAR T cells and did not significantly affect CAR T cell expansion and cell phenotype. HHV-6 reactivation in primary PBMCs may occur during the manufacturing process with extended cell culture, but is nevertheless an extremely rare event. We desired to understand whether the rare event of reactivation can be prevented by adding an antiviral agent, such as IFNα, at a specific timepoint during the manufacturing process. To mimic the rare reactivation event, PBMC-derived CAR T cells were infected with exogenous HHV-6 at an MOI of 0.4 on Day 8 of the small scale manufacturing process. Cells were treated with IFNα at a concentration of 1 ng/μL or 10 ng/μL on Day 0 (preactivation), Day 1 (activation), Day 4, or Day 6, or untreated throughout the process. All cultures were infected with HHV-6 on Day 8, regardless whether the cells were pre-treated with IFNα or left untreated. A summary of treatment conditions is shown in Table 1 below:

TABLE 1

| Condition | IFNα Added on | IFNα Concentration |
|---|---|---|
| C1 | Day 0 | 10 ng/μL |
| C2 | Day 0 | 1 ng/μL |
| C3 | Day 1 | 10 ng/μL |
| C4 | Day 1 | 1 ng/μL |
| C5 | Day 4 | 10 ng/μL |
| C6 | Day 4 | 1 ng/μL |
| C7 | Day 6 | 10 ng/μL |
| C8 | Day 6 | 1 ng/μL |
| C9 | None | N/A |

The effects of IFNα on HHV-6 infection were analyzed in cells from two different donors. The transcripts of viral genes U100 (FIGS. 6A and 7A) and U79 (FIGS. 6B and 7B) were analyzed by RT-qPCR at the time points of three, seven and ten days post infection (i.e., on Day 11, Day 15 and Day 18 of the small scale manufacturing process). The y-axis reprepresents relative-fold change for each value compared to the endogenous GAPDH as well as untreated donor RNA (Day 0 before IFNα treatment). The results in FIG. 6A show that IFNα added on Day 6, i.e., two days before infection (experiment conditions C7 and C8) led to the greatest reduction of U100 transcript level compared to the sample without IFNα pre-treatment (C9), as well as the lowest level of viral load as determined by qPCR (data not shown). The C7 and C8 samples shown in FIG. 6A also had the lowest fold-change of U100 trranscript compared to the baseline of Day 0. In this particular experiment, the C5 and C6 samples shown in FIG. 6A had fewer cells available for analysis for reasons unknown. When the transcript of another viral gene U79 was analyzed, all samples C5-C8 showed decreased transcript (FIG. 6B). The same experiments were repeated in cells from a different donor #3 (FIG. 7A and FIG. 7B). The data of FIGS. 6A-B and FIGS. 7A-B showed generally an IFNα dose-dependent response, but some donor-specific variability was also observed. The data suggest that IFNα treatment at a timepoint close to HHV-6 infection (a surrogate or approximation of HHV-6 reactivation in this experiment), such as Day 4 or Day 6 of the cell culture after activation on Day 1, can be most effective in controling HHV-6 reactivation in the manufacturing process.

The viral DNA replication was also analyzed by qPCR of the DNA of viral gene U31 and the results suggest that doses of IFNα added closer to infection (a surrogate for reactivation) had the strongest suppression effect on HHV-6 infection (data not shown).

### Example 7 Effects of Additional Antiviral Agents on PBMCs with Integrated HHV-6 Viral DNA

We next examined the effects of other known antiviral agents on controlling HHV-6 reactivation using PBMCs with integrate HHV-6 genomes (iciHHV6) to mimic continuous low levels of HHV-6 infection. Ganciclovir, cidofovir and foscarnet are all small molecule inhibitors of HHV viral DNA polymerase. Artesunate is a small molecule antiviral agent targeting HHV immediate early genes. All four agents were in clinical use and have demonstrated in vitro anti-HHV-6 activity. See e.g., hhv-6foundation.org/research/hhv-6-antiviral-drug-resistance; Reyman et al., 1995, Antiviral Res. 28:343-57; and Vittayawacharin et al., 2023, Transplant Cell Ther. 29:397. The cells were cultured in X-vivo supplemented media for three days before being split into 4 subcultures. Different antiviral agents were added on day 4 at a concentration as shown in Table 2. Afterwards, cells were cultured for 15 additional days, with media exchange twice a week, each time containing a fresh dose of the same antiviral agent at the same initial concentration. Cell sampling was taken on different days after the antiviral agent was first added. The effect of the antiviral agent on the viability of cells were analyzed by using a cellular apoptosis screening kit (Thermo Cat #V13242),.

TABLE 2

| | | EC50 | | Concentration | |
|---|---|---|---|---|---|
| | | (Reyman | | for use in | |
| Drug Name | Vendor and Cat # | 1995) | | experiment | |
| Ganciclovir | Selleckchem S1878 | >25 | μM | 25 | μM |
| Cidofovir | Selleckchem S1516 | 12-15 | μM | 13 | μM |
| Foscarnet | Selleckchem S3076 | 8.4 | μM | 8.4 | μM |
| Artesunate | Selleckchem S2265 | 3.8 | μM | 3.8 | μM |

The data in FIG. 8 show that foscarnet and ganciclovir at the concentrations tested did not negatively affect cell health. On the other hand, artusunate, and to a less extent cidofovir at the concentrations tested induced higher level of apoptosis as compared to the other antiviral agents. Next, HHV-6 replication was analyzed by detecting copies of HHV-6 gene U65 by qPCR. The data in FIG. 9 show that foscarnet was able to maintain the HHV-6 levels at around their starting level, i.e. preventing any further HHV-6 expansion. Ganciclovir showed both a decreased viral load initially followed by an increase at later time points, suggesting that concentrations higher than 25 μM can be more effective. In contrast, cells treated with artusunate and cidofovir showed increased HHV-6 levels over the culture period, suggesting that the concentrations of drug tested were not potent enough to suppress the HHV-6 expansion. The results indicate that, in the cell culture model, foscarnet is the most effective antiviral agent on controlling HHV6, followed by ganciclovir, and cidofovir potentially when used at higher concentrations.

### Materials & Methods

DNA qPCR

Genomic DNA was extracted from cells and DNA levels of HHV-6 were determined by quantitative PCR (qPCR). The experiment was carried out using the ThermoFisher Scientific, TaqMan™ Fast Virus 1-Step Master Mix, (Catalog number: 4444434). Primers and probes against these genes are shown in Table 3.

TABLE 3
qPCR Primer/Probe Set Sequences

| | | SEQ ID |
|---|---|---|
| | Sequence | NO: |
| HHV-6 U31 | Forward primer (5′-3′) | 1 |
| | CGACTCTCACCCTACTGAACGA | |
| | Reverse primer (5′-3′) | 2 |
| | GAGGCTGGCGTCGTAGTAGAA | |
| | Probe (5′-3′), YakYel at 5′ end and 3IABKFQ | 3 |
| | at 3′ end | |
| | AGCCACAGC/ZEN/AGCCATCTACATCTGTCAA | |
| HHV-6 U65-66 | Forward primer (5′-3′) | 4 |
| | GACAATCACATGCCTGGATAATG | |
| | Reverse primer (5′-3′) | 5 |
| | TGTAAGCGTGTGGTAATGGACTA | |
| | Probe (5′-3′), 6-FAM and 3IABKFQ at 3′ end | 6 |
| | AGCAGCTGG/ZEN/CGAAAAGTGCTGTGC | |

### RNA RT-qPCR

Total RNA was extracted from cells and analyzed using reverse transcription qPCR (RT-qPCR) assay for U79, U90, U100 and GAPDH genes to determine HHV-6 infectivity. Primers and probes against these genes are shown in Table 4.

TABLE 4
RT-qPCR Primer/Probe Set Sequences

| | Sequence | SEQ ID NO: |
|---|---|---|
| HHV-6 U79 | Forward primer (5′-3′) | 7 |
| | TTTCGTTTCGTGGTGAGATTT | |
| | Reverse primer (5′-3′) | 8 |
| | AAAGTGGAGGACATTGACAAA | |
| | Probe (5′-3′), 6-FAM at 5′ end and 3IABKFQ | 9 |
| | at 3′ end | |
| | ATGAATTTG/ZEN/ATGAACCCCCTAAGGAG | |
| HHV-6 U90 | Forward primer (5′-3′) | 10 |
| | TTTCATGGCAGCCTTCACT | |
| | Reverse primer (5′-3′) | 11 |
| | ATGTGGAAGAAGGAGCATCTG | |
| | Probe (5′-3′), 6-FAM and 3IABKFQ at 3′ end | 12 |
| | CAAAACTTT/ZEN/GGTTGCTCAGTCCGTTG | |
| HHV-6 U100 | Forward primer (5′-3′) | 13 |
| | TGACTAGTAAATTGGAGCTACGC | |
| | Reverse primer (5′-3′) | 14 |
| | GTCTGTCCGCCATGGTTT | |
| | Probe (5′-3′) 6-FAM and 3IABKFQ at 3′ end | 15 |
| | AAAGGCTCG/ZEN/ATGCGAACTGATCGT | |
| Human GAPDH | Forward (5′-3′) | 16 |
| | GTGGTCTCCTCTGACTTCAAC | |
| | Reverse (5′-3′) | 17 |
| | CCTGTTGCTGTAGCCAAATTC | |
| | Probe (5′-3′) 6-FAM and 3IABKFQ at 3′ end | 18 |
| | TTGCCCTCA/ZEN/ACGACCACTTTGTCA | |

### FACS

HHV6 infectivity was determined by intracellular staining using antibody against HHV-6 p41 protein at 1 μg/mL and FACS analysis was performed to determine the percent of cells expressing the viral protein.

% CAR+ and CAR-T cell phenotype were determined by staining cells for the following markers CD45, CD5, CD4, CD8, CAR. To identify memory T cells phenotypes, CD45RA and CD62L markers were used, e.g., T_{eff} (CD45RA+CD62L−), Tₑₘ (CD45RA-CD62L−), T_{cm} (CD45RA-CD62L+) and T_{scm} (CD45RA+CD62L+)., etc.

### Immunofluorescence

The cells were fixed with 4% paraformaldehyde and permeabilized using 0.5% saponin before incubating with HHV6 p41 antibody conjugated with Alexa-488 at 0.2 μg/mL. Cells were stained with DAPI prior to imaging at 20× magnification.

What is claimed is:

1. A method of preventing or inhibiting HHV-6 replication, preventing or inhibiting HHV-6 transcription activation, preventing or inhibiting HHV-6 infection, or preventing or inhibiting HHV-6 replication or infection after reactivation in a process of making engineered immune cells, the method comprising the steps of culturing immune cells, engineering the immune cells, and contacting the immune cells with an antiviral agent in a culture medium, optionally wherein the immune cells are suspected to have latent HHV-6 infection.

2. The method of claim 1, wherein the step of contacting the immune cells with the antiviral agent occurs by adding the antiviral agent to the culture medium.

3. The method of claim 2, wherein the antiviral agent is added to the culture medium on day 4 to day 10, day 5 to day 10, day 6 to day 10, day 7 to day 10, day 8 to day 10, or day 9 to day 10 of the process of making the engineered immune cells.

4. The method of claim 2 or 3, wherein the antiviral agent is added to the cell culture medium after the cells have been cultured for at least 4 days, at least 5 days, at least 6 days, at least 7 days, at least 8 days, or at least 9 days.

5. The method of any one of claims 2-4, wherein the antiviral agent is added to the cell culture medium 3 days, 4 days, 5 days, 6 days, 7 days, or 8 days after the immune cells have been activated, optionally, the immune cells are activated by an anti-CD3 antibody and an anti-CD28 antibody.

6. The method of any one of the preceding claims, wherein the immune cells are contacted with the antiviral agent for about 2 days, about 3 days, about 4 days, or about 5 days.

7. The method of any one of the preceding claims, wherein the antiviral agent is interferon (IFN), foscarnet or ganciclovir.

8. The method of any one of the preceding claims, wherein the antiviral agent is a type I IFN.

9. The method of any one of the preceding claims, wherein the antiviral agent is a type III IFN.

10. The method of any one of the preceding claims, wherein the antiviral agent is IFN, and wherein the IFN is added to the culture medium at a concentration of about 0.1 ng/ml to about 100 ng/ml or about 0.1 ng/ml to about 10 ng/ml.

11. The method of any one of the preceding claims, wherein the antiviral agent is IFN, and wherein the IFN is added to the culture medium at a concentration of about 0.1 ng/ml to about 1 ng/ml.

12. The method of any one of the preceding claims, wherein the antiviral agent is foscarnet, and wherein foscarnet is added to the culture medium at a concentration of about 8 μM to about 15 μM.

13. The method of any one of the preceding claims, wherein the antiviral agent is ganciclovir, and wherein the ganciclovir is added to the culture medium at a concentration of 25 μM to about 35 μM.

14. The method of any one of the preceding claims, wherein the engineered immune cells are T cells, PBMCs, IPSCs or NK cells.

15. The method of any one of the preceding claims, wherein the engineered immune cells exhibit reduced levels of HHV-6 replication, HHV-6 transcription activation, HHV-6 infection, or HHV-6 replication or infection after reactivation, as compared to control engineered immune cells without being contacted with the antiviral agent.

16. The method of any one of the preceding claims, wherein the engineered immune cells exhibited comparable levels of combined Tcm and Tscm as compared to control engineered immune cells without being contacted with the antiviral agent.

17. The method of any one of the preceding claims, further comprising the step of detecting or measuring HHV-6 DNA levels, RNA levels or protein levels during the process of making the engineered immune cells.

18. The method of any one of the preceding claims, further comprising a step of detecting or measuring HHV-6 DNA levels on day one of the process of making the engineered immune cells, wherein the HHV-6 DNA levels measured during the process of making the engineered immune cells are comparable to the HHV-6 DNA levels measured on day one of the process of making the engineered immune cells.

19. The method of any one of the preceding claims, further comprising the step of detecting HHV-6 RNA levels during the process of making the engineered immune cells.

20. The method of any one of claims 17-19, wherein the HHV-6 DNA levels, RNA levels or protein levels are determined by PCR, qPCR, RT-PCR, RT-qPCR, ELISA, immunofluorescent assay, or flow cytometry.

21. The method any one of the preceding claims, wherein the step of engineering the immune cells comprises introducing to the immune cells an exogenous polynucleotide that encodes a chimeric antigen receptor (CAR) or recombinant T cell receptor (TCR).

22. The method of claim 21, wherein the exogenous polynucleotide is introduced to the immune cells by lentiviral transduction or by adenovirus associated viral transduction.

23. The method of any one of the preceding claims, wherein the step of engineering the immune cells further comprises modifying one or both TCRα genetic loci to reduce or eliminate the expression or activity of the TCRα gene.

24. The method of any one of claims 1-22, wherein the immune cells are obtained from a patient.

25. The method of any one of claims 1-23, wherein the immune cells are obtained from a healthy donor.

26. The method of any one of the preceding claims, wherein the immune cells harbor latent infection of HHV-6.

27. The method of any one of the preceding claims, wherein the antiviral agent is human IFNα.

28. An engineered immune cell produced by the method of any one of claims 1-27.

29. A population of engineered immune cells of claim 28.

30. A method for generating an in vitro cell culture model of HHV-6 latent infection and reactivation comprising the steps of
(a) infecting human lymphoid cells with HHV-6;
(b) culturing the infected cells for about 12 to about 19 days; and
(c) serially passaging the cells to maintain a cell density of about 0.2-0.8×10⁶ cells per cm² for about 30 days to about 60 days, thereby establishing latent HHV-6 infection in the cells.

31. The method of claim 30, further comprising detecting HHV-6 viral DNA or detecting HHV-6 viral RNA, after serial passaging of the cells in step (c), wherein a constant level of HHV-6 DNA or an absence of detectable RNA indicates latent infection.

32. The method of claim 30 or 31, wherein the latent HHV-6 infection in the cells can be reactivated by a stimulant.

33. The method of claim 32, wherein the stimulant is sodium butyrate or PMA.

34. An in vitro cell culture model for HHV-6 latent infection generated by the method of claim 30, wherein no more than 10⁴ copies of HHV-6 viral genome equivalents per 500 ng extracted DNA can be detected by qPCR in the cells and/or no HHV-6 transcript can be detected by RT-qPCR in the cells.

35. The in vitro cell culture model of claim 34, wherein active HHV-6 infection can be induced or reactivated in the cells.

36. The in vitro cell culture model of claim 34 or 35, wherein the active HHV-6 infection can be induced or reactivated by contacting the cells with sodium butyrate and PMA.

37. The in vitro cell culture model of claim 35 wherein the active HHV-6 infection is demonstrated by detection of one or more HHV-6 transcripts by RT-PCR or RT-qPCR.

38. An in vitro cell culture model for HHV-6 latent infection, wherein no more than 10⁴ copies of HHV-6 viral genome equivalents per 500 ng extracted DNA can be detected by qPCR in the cells and/or no HHV-6 transcript can be detected by RT-qPCR in the cells, wherein the cells are human lymphoid cells.

39. The in vitro cell culture model of claim 38, wherein active HHV-6 infection can be induced or reactivated in the cells.

40. The in vitro cell culture model of claim 39, wherein the active HHV-6 infection can be induced or reactivated by contacting the cells with sodium butyrate and PMA.

41. The in vitro cell culture model of claim 39 or 40 wherein the active HHV-6 infection is demonstrated by detection of HHV-6 transcripts by RT-PCR or RT-qPCR.

42. A method of making engineered immune cells comprising the steps of (a) culturing immune cells; (b) engineering the immune cells; and (c) contacting the immune cells with an antiviral agent in a culture medium, wherein the immune cells comprise latent HHV-6 infection.

43. The method of claim 42, wherein the step of contacting the immune cells with the antiviral agent occurs by adding the antiviral agent to the culture medium.

44. The method of claim 42 or 43, wherein the antiviral agent is added to the culture medium on day 4 to day 10, day 5 to day 10, day 6 to day 10, day 7 to day 10, day 8 to day 10, or day 9 to day 10 after culturing the immune cells.

45. The method of any one of claims 42-44, wherein the antiviral agent is added to the cell culture medium after the cells have been cultured for at least 4 days, at least 5 days, at least 6 days, at least 7 days, at least 8 days, or at least 9 days.

46. The method of any one of claims 42-45, wherein the antiviral agent is added to the cell culture medium 3 days, 4 days, 5 days, 6 days, 7 days, or 8 days after the immune cells have been activated, optionally, the immune cells are activated by an anti-CD3 antibody and an anti-CD28 antibody.

47. The method of any one of claims 42-46, wherein the immune cells are contacted with the antiviral agent for about 2 days, about 3 days, about 4 days, or about 5 days.

48. The method of any one of claims 42-47, wherein the antiviral agent is a type I IFN or a type III IFN.

49. The method of any one of claims 42-48, wherein the antiviral agent is IFN and the IFN is added to the culture medium at a concentration of about 0.1 ng/ml to about 100 ng/ml or about 0.1 ng/ml to about 10 ng/ml.

50. The method of any one of claims 42-49, wherein the IFN is added to the culture medium at a concentration of about 0.1 ng/ml to about 1 ng/ml.

51. The method of any one of claims 42-50, wherein the antiviral agent is foscarnet, and wherein foscarnet is added to the culture medium at a concentration of about 8 μM to about 15 μM.

52. The method of any one of claims 42-51, wherein the antiviral agent is ganciclovir, and wherein the ganciclovir is added to the culture medium at a concentration of 25 μM to about 35 μM

53. The method of any one of claims 42-52, wherein the immune cells are T cells, PBMCs, iPSCs or NK cells.

54. The method of any one of claims 42-53, further comprising the step of detecting or measuring HHV-6 DNA levels, RNA levels or protein levels after the step of contacting the immune cells with the antiviral agent.

55. The method of claim 54, further comprising a step of detecting or measuring HHV-6 DNA levels before contacting the immune cells with the antiviral agent, wherein the HHV-6 DNA levels measured after contacting the immune cells with the IFN are comparable to the HHV-6 DNA levels measured before the step of contacting the immune cells with the antiviral agent.

56. The method of claim 54, further comprising the step of detecting HHV-6 RNA levels, wherein HHV-6 RNA levels are not detectable.

57. The method of any one of claims 54-56, wherein the HHV-6 DNA levels, RNA levels or protein levels are determined by PCR, qPCR, RT-PCR, RT-qPCR, ELISA, immunofluorescent assay, or flow cytometry.

58. The method any one of claims 42-57, wherein the step of engineering the immune cells comprises introducing to the immune cells an exogenous polynucleotide that encodes a chimeric antigen receptor (CAR) or recombinant T cell receptor (TCR).

59. The method of claim 58, wherein the exogenous polynucleotide is introduced to the immune cells by lentiviral transduction or by adenovirus associated viral transduction.

60. The method of any one of claims 42-59, wherein the step of engineering the immune cells further comprises modifying one or both TCRα genetic loci to reduce or eliminate the expression or activity of the TCRα gene.

61. The method of any one of claims 42-59 wherein the immune cells are obtained from a patient.

62. The method of any one of claims 38-60, wherein the immune cells are obtained from a healthy donor.

63. The method of any one of claims 48-62, wherein the IFN is human IFNα.


SEQUENCE LISTING (20 sequences)

SEQ ID NO: 1 - DNA, 22 bp, synthetic construct
  misc_feature 1..22: note = Description of sequence: HHV-6 U31 Forward primer
  source 1..22: mol_type = other DNA
cgactctcaccctactgaacga

SEQ ID NO: 2 - DNA, 21 bp, synthetic construct
  misc_feature 1..21: note = Description of sequence: HHV-6 U31 Reverse primer
  source 1..21: mol_type = other DNA
gaggctggcgtcgtagtagaa

SEQ ID NO: 3 - DNA, 29 bp, synthetic construct
  misc_feature 1..29: note = Description of sequence: HHV-6 U31 Probe YakYel at 5' end and 3IABkFQ at 3' end
  source 1..29: mol_type = other DNA
  modified_base 10: mod_base = OTHER; note = n is N,N-diethyl-4-(4-nitronaphthalen-1-ylazo)-phenylamine
  modified_base <1: mod_base = OTHER; note = Yakima yellow dye attached
  modified_base >29: mod_base = OTHER; note = Iowa Black FQ dye attached
agccacagcnagccatctacatctgtcaa

SEQ ID NO: 4 - DNA, 23 bp, synthetic construct
  misc_feature 1..23: note = Description of sequence: HHV-6 U65-66 Forward primer
  source 1..23: mol_type = other DNA
gacaatcacatgcctggataatg

SEQ ID NO: 5 - DNA, 23 bp, synthetic construct
  misc_feature 1..23: note = Description of sequence: HHV-6 U65-66 Reverse primer
  source 1..23: mol_type = other DNA
tgtaagcgtgtggtaatggacta

SEQ ID NO: 6 - DNA, 25 bp, synthetic construct
  misc_feature 1..25: note = Description of sequence: HHV-6 U65-66 Probe 6-FAM at 5' end and 3IABkFQ at 3' end
  source 1..25: mol_type = other DNA
  modified_base 10: mod_base = OTHER; note = n is N,N-diethyl-4-(4-nitronaphthalen-1-ylazo)-phenylamine
  modified_base <1: mod_base = OTHER; note = 6-FAM Fluorescent label
  modified_base >25: mod_base = OTHER; note = Iowa Black FQ dye attached
agcagctggncgaaaagtgctgtgc

SEQ ID NO: 7 - DNA, 21 bp, synthetic construct
  misc_feature 1..21: note = Description of sequence: HHV-6 U79 Forward primer
  source 1..21: mol_type = other DNA
tttcgtttcgtggtgagattt

SEQ ID NO: 8 - DNA, 21 bp, synthetic construct
  misc_feature 1..21: note = Description of sequence: HHV-6 U79 Reverse primer
  source 1..21: mol_type = other DNA
aaagtggaggacattgacaaa

SEQ ID NO: 9 - DNA, 27 bp, synthetic construct
  misc_feature 1..27: note = Description of sequence: HHV-6 U79 Probe 6-FAM at 5' end and 3IABkFQ at 3' end
  source 1..27: mol_type = other DNA
  modified_base 10: mod_base = OTHER; note = n is N,N-diethyl-4-(4-nitronaphthalen-1-ylazo)-phenylamine
  modified_base <1: mod_base = OTHER; note = 6-FAM Fluorescent label
  modified_base >27: mod_base = OTHER; note = Iowa Black FQ dye attached
atgaatttgnatgaaccccctaaggag

SEQ ID NO: 10 - DNA, 19 bp, synthetic construct
  misc_feature 1..19: note = Description of sequence: HHV-6 U90 Forward primer
  source 1..19: mol_type = other DNA
tttcatggcagccttcact

SEQ ID NO: 11 - DNA, 21 bp, synthetic construct
  misc_feature 1..21: note = Description of sequence: HHV-6 U90 Reverse primer
  source 1..21: mol_type = other DNA
atgtggaagaaggagcatctg

SEQ ID NO: 12 - DNA, 27 bp, synthetic construct
  misc_feature 1..27: note = Description of sequence: HHV-6 U90 Probe 6-FAM at 5' end and 3IABkFQ at 3' end
  source 1..27: mol_type = other DNA
  modified_base 10: mod_base = OTHER; note = n is N,N-diethyl-4-(4-nitronaphthalen-1-ylazo)-phenylamine
  modified_base <1: mod_base = OTHER; note = 6-FAM Fluorescent label
  modified_base >27: mod_base = OTHER; note = Iowa Black FQ dye attached
caaaactttnggttgctcagtccgttg

SEQ ID NO: 13 - DNA, 23 bp, synthetic construct
  misc_feature 1..23: note = Description of sequence: HHV-6 U100 Forward primer
  source 1..23: mol_type = other DNA
tgactagtaaattggagctacgc

SEQ ID NO: 14 - DNA, 18 bp, synthetic construct
  misc_feature 1..18: note = Description of sequence: HHV-6 U100 Reverse primer
  source 1..18: mol_type = other DNA
gtctgtccgccatggttt

SEQ ID NO: 15 - DNA, 25 bp, synthetic construct
  misc_feature 1..25: note = Description of sequence: HHV-6 U100 Probe 6-FAM at 5' end and 3IABkFQ at 3' end
  source 1..25: mol_type = other DNA
  modified_base 10: mod_base = OTHER; note = n is N,N-diethyl-4-(4-nitronaphthalen-1-ylazo)-phenylamine
  modified_base <1: mod_base = OTHER; note = 6-FAM Fluorescent label
  modified_base >25: mod_base = OTHER; note = Iowa Black FQ dye attached
aaaggctcgnatgcgaactgatcgt

SEQ ID NO: 16 - DNA, 21 bp, synthetic construct
  misc_feature 1..21: note = Description of sequence: Human GAPDH Forward
  source 1..21: mol_type = other DNA
gtggtctcctctgacttcaac

SEQ ID NO: 17 - DNA, 21 bp, synthetic construct
  misc_feature 1..21: note = Description of sequence: Human GAPDH Reverse
  source 1..21: mol_type = other DNA
cctgttgctgtagccaaattc

SEQ ID NO: 18 - DNA, 25 bp, synthetic construct
  misc_feature 1..25: note = Description of sequence: Human GAPDH Probe 6-FAM at 5' end and 3IABkFQ at 3' end
  source 1..25: mol_type = other DNA
  modified_base 10: mod_base = OTHER; note = n is N,N-diethyl-4-(4-nitronaphthalen-1-ylazo)-phenylamine
  modified_base <1: mod_base = OTHER; note = 6-FAM Fluorescent label
  modified_base >25: mod_base = OTHER; note = Iowa Black FQ dye attached
ttgccctcanacgaccactttgtca

SEQ ID NO: 19 - protein, 188 aa, Homo sapiens
  REGION 1..188: note = Description of sequence: Human interferon alpha 2a precursor protein)
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 20 - protein, 165 aa, Homo sapiens [sequence omitted]